(19) United States
(12) Patent Application Publication
(10) Pub. No.: US 2008/0289124 A1
(43) Pub. Date: Nov. 27, 2008

(54) Reactive Azo Dyes, Methods for the Production Thereof and Their Use

(75) Inventor: Gunter Gorlitz, Sulzbach/Ts. (DE)

Correspondence Address: CONNOLLY BOVE LODGE & HUTZ, LLP, P O BOX 2207, WILMINGTON, DE, 19899, US

(73) Assignee: DyStar Textilfarben Gmbh & Co. Deutschland KG, Frankfurt am Main (DE)

(21) Appl. No.: 11/912,441

(22) PCT Filed: Apr. 19, 2006
(86) PCT No.: PCT/EP2006/061657

§ 371 Date: May 8, 2008

(30) Foreign Application Priority Data
Apr. 26, 2005 (DE) 10 2005 019 333.1

(51) Int. Cl. C09B 62/405; C09B 29/42; C07D 213/72

(52) U.S. Cl. 8/543; 534/773; 546/307

(57) ABSTRACT

Reactive dyes of the formula (1)

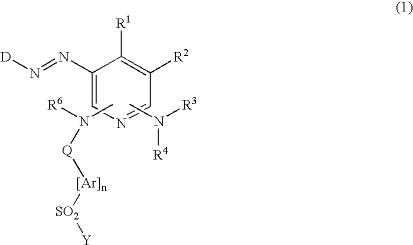

where R¹ to R⁵, Ar, n, Y and D are as defined in claims 1 and 2 are described, as are processes for preparing them, and their use for dyeing and printing carboxamido-, amino-, and hydroxyl-containing material.

The present invention is situated within the field of fiber-reactive dyes. Documents DE 3 025 904, EP 0 581 730 and EP 0 581 731 have already disclosed dyes which have structural similarities to the dyes of the invention, described below, but which differ with regard to the reactive “anchor” in the coupling component. In the context of the dyeing of textile materials, these known dyes have a number of technical disadvantages, which require amelioration.

Surprisingly it has now been found that the dyes below of the formula (1) are advantageous over the known dyes.

The present invention provides reactive dyes of the formula (1)

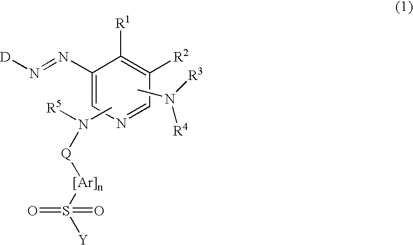

in which
D is the radical of a diazo component of the benzene or naphthalene series, a heterocyclic radical or the radical of a monoazo or diazo dye,
R¹ is C₁-C₄-alkyl,
R² is cyano, carbamoyl or sulfomethyl,
R³, R⁴ and R⁵ independently of one another are hydrogen or are C₁-C₁₈-alkyl or C₄-C₇-cycloalkyl each of which is unsubstituted or substituted by hydroxyl, sulfo, sulfato or vinylsulfonyl and with the exception of methyl is uninterrupted or interrupted by oxygen or NR⁷, in which R⁷ is hydrogen or C₁-C₄-alkyl, or R³ and R⁴ are part of heterocyclic groups,
Q is C₁-C₁₈-alkylene which is unsubstituted or substituted by C₁-C₄-alkyl, halogen or hydroxyl and, with the exception of methylene, uninterrupted or interrupted by oxygen or NR⁷,
n is 0 or 1,
Ar is a phenylene or naphthylene radical which is unsubstituted or substituted by sulfo, carboxyl, C₁-C₄-alkyl, C₁-C₄-alkoxy or halogen,
Y is —CH═CH₂ or —CH₂CH₂Y¹, in which
   Y¹ is hydroxyl or a group which can be eliminated under the action of alkali.

In the dyes of the invention D is preferably a group of the formula (D-I), (D-II) or (D-XII)

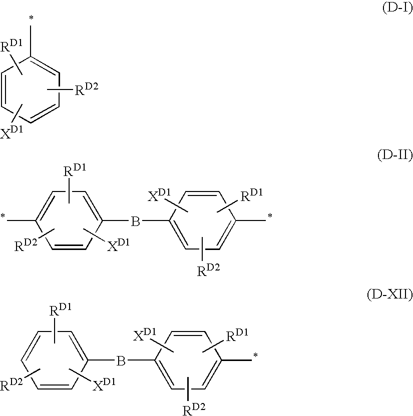

where
-* is the bond to the diazo group on the diaminopyridine group,
B is a direct chemical bond, an ethylene group or a diazo group,
R^{D1} and R^{D2} independently of one another are hydrogen, (C₁-C₄)-alkyl, (C₁-C₄)-alkoxy, hydroxyl, sulfo, carboxyl, cyano, nitro, amido, ureido or halogen, and
X^{D1} has one of the definitions of R^{D1} or R^{D2} or is a group of the formula —SO₂-Z,
   where
   Z has one of the definitions of Y, or
D is a naphthyl group of the formula (D-III)

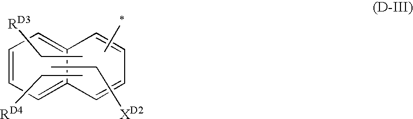

in which
-* is the bond of the diazo group on the diaminopyridine group,
R^{D3} and R^{D4} independently of one another are hydrogen, (C₁-C₄)-alkyl, (C₁-C₄)-alkoxy, hydroxyl, sulfo, carboxyl, cyano, nitro, amido, ureido or halogen, and
X^{D2} has one of the definitions of X^{D1}; or
D is a group of the formula (D-IV)

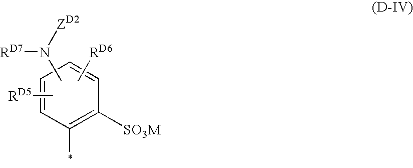

in which
-* is the bond to the diazo group on the diaminopyridine group,
M is hydrogen, an alkali metal, ammonium, one equivalent of an alkaline earth metal, or a monovalent organic cation, especially alkylammonium,
R^{D5} and R^{D6} independently of one another have one of the definitions of R^{D1} and R^{D2};
R^{D7} is hydrogen, (C₁-C₄)-alkyl, or phenyl unsubstituted or substituted by (C₁-C₄)-alkyl, (C₁-C₄)-alkoxy, sulfo, halogen or carboxyl, and
Z^{D2} is a group of the formula (D-V), (D-VI) or (D-VII)

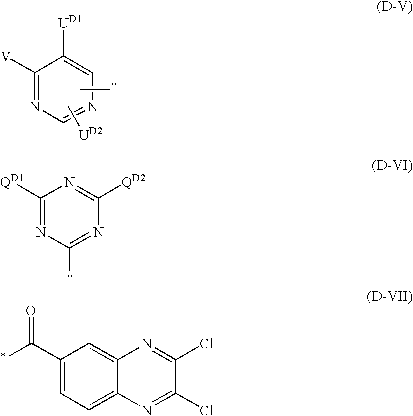

in which
-* is the bond to nitrogen,
   V is fluorine or chlorine,
   U^{D1} and U^{D2} independently of one another are fluorine, chlorine or hydrogen, and
   Q^{D1} and Q^{D2} independently of one another are chlorine, fluorine, cyanamide, hydroxyl, (C₁-C₆)-alkoxy, phenoxy, sulfophenoxy, mercapto, (C₁-C₆)-alkylmercapto, pyridino, carboxypyridino, carbamoylpyridino or a group of the formula (D-VIII) or (D-IX)

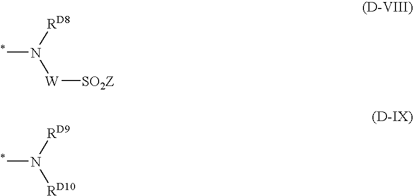

in which
-* is the bond to the heterocycle,
   R^{D8} is hydrogen or (C₁-C₆)-alkyl, sulfo-(C₁-C₆)-alkyl, or phenyl which is unsubstituted or substituted by (C₁-C₄)-alkyl, (C₁-C₄)-alkoxy, sulfo, halogen, carboxyl, acetamido or ureido;
   R^{D9} and R^{D10} independently of one another have one of the definitions of R^{D8} or form a cyclic ring system of the formula —(CH₂)ⱼ—, where j is 4 or 5, or alternatively —(CH₂)₂-E-(CH₂)₂—, where E is oxygen, sulfur, sulfo or —NR^{D11} and R^{D11} is (C₁-C₆)-alkyl;
W is phenylene which is unsubstituted or substituted by 1 or 2 substituents, such as (C₁-C₄)-alkyl, (C₁-C₄)-alkoxy, carboxyl, sulfo, chlorine or bromine, or
W is (C₁-C₄)-alkylenearylene or (C₂-C₆)-alkylene which may be interrupted by oxygen, sulfur, sulfo, amino, carbonyl or carboxamido, or
W is phenylene-CONH-phenylene, which is unsubstituted or substituted by (C₁-C₄)-alkyl, (C₁-C₄)-alkoxy, hydroxyl, sulfo, carboxyl, amido, ureido, or halogen, or
W is naphthylene which is unsubstituted or substituted by one or two sulfo groups, and
   Z is as defined above, or
D is a group of the formula (D-X) or (D-XI)

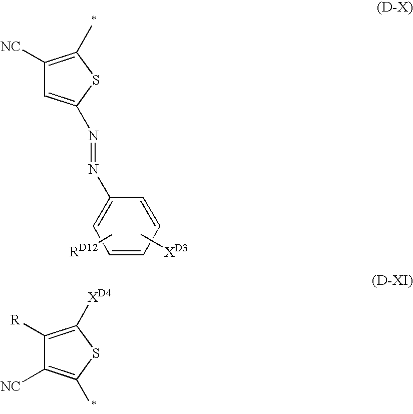

where
-* is the bond to the diazo group on the diaminopyridine group,
R^{D12} has one of the definitions of R^{D1},
X^{D3} has one of the definitions of X^{D1},
X^{D4} is —CN or COOR^{D13}, where R¹³ has one of the definitions of R and R is C₁-C₄-alkyl.

Alkali-eliminable substitutents Y¹ in the β position of the ethyl group of Y are for example halogen atoms, such as chlorine and bromine, ester groups of organic carboxylic and sulfonic acids, such as alkylcarboxylic acids, unsubstituted or substituted benzenecarboxylic acids and unsubstituted or substituted benzenesulfonic acids, such as the alkanoyloxy groups of 2 to 5 carbon atoms, especially acetyloxy, benzoyloxy, sulfobenzoyloxy, phenylsulfonyloxy and tolylsulfonyloxy, and also acidic ester groups of inorganic acids, such as of phosphoric acid, sulfuric acid and thiosulfuric acid (phosphato, sulfato and thiosulfato groups), and also dialkylamino groups having alkyl groups each of 1 to 4 carbon atoms, such as dimethylamino and diethylamino, and unsubstituted or substituted pyridinium, nicotinato and isonicotinato.

Y is preferably β-sulfatoethyl or β-chloroethyl, and more preferably vinyl.

The groups “sulfo”, “carboxyl”, “thiosulfato”, “phosphato”, and “sulfato”, include not only their acid form but also their salt form. Hence sulfo groups are groups of the formula —SO₃M, thiosulfato groups are groups of the is formula —S—SO₃M, carboxyl groups are groups of the formula —COOM, phosphato groups are groups of the formula —OPO₃M₂, and sulfato groups are groups of the formula —OSO₃M, with M being hydrogen, an alkali metal, ammonium, one equivalent of an alkaline earth metal, or monovalent organic cations, especially alkylammonium.

Preferred reactive dyes of the invention are those of the formula (1a)

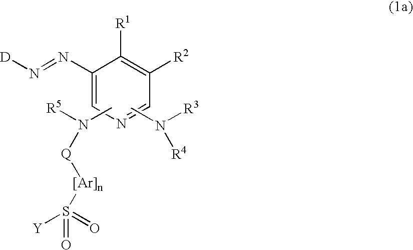

in which
(D-I), Ar and R¹ to R⁵ are as defined above and
n is 0 or 1.

Additionally preferred reactive dyes of the invention are those of the formula (1b)

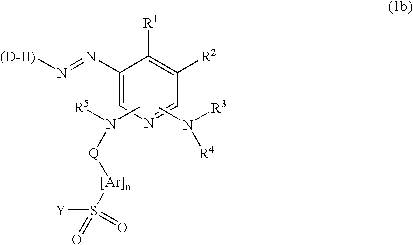

in which
(D-II), Ar and R¹ to R⁵ are as defined above and
n is 0 or 1.

Additionally preferred reactive dyes of the invention are those of the formula (1c)

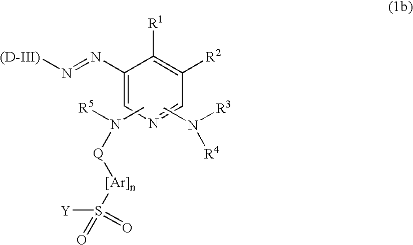

in which
(D-II), Ar and R¹ to R⁵ are as defined above and
n is 0 or 1.

Especially preferred reactive dyes of the invention are those of the formula (1d)

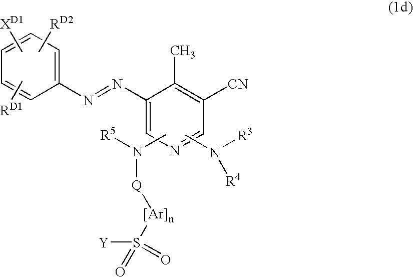

in which
X^{D1} has one of the definitions of R^{D1} or R^{D2} and R³ and R⁴, Ar, Q and Y are as defined above, and
R⁵ is hydrogen or C₁-C₁₈-alkyl.

Additionally, especially preferred reactive dyes of the invention are of the formula (1e)

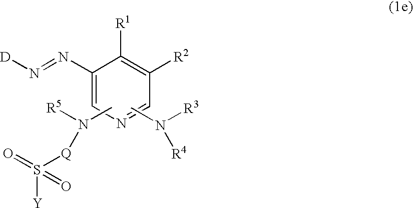

in which
R¹ is C₁-C₄-alkyl,
R² is cyano, carbamoyl or sulfomethyl,
R³ and R⁴ independently of one another are hydrogen or are C₁-C₁₈-alkyl or C₄-C₇-cycloalkyl each of which is unsubstituted or substituted by hydroxyl, sulfo, sulfato or vinylsulfonyl and with the exception of methyl is uninterrupted or interrupted by oxygen or NR⁷, in which R⁷ is hydrogen or C₁-C₄-alkyl, or R³ and R⁴ are part of heterocyclic groups,
R⁵ is hydrogen or C₁-C₁₈-alkyl,
Q is C₁-C₁₈-alkylene which is unsubstituted or substituted by C₁-C₄-alkyl, halogen or hydroxyl and with the exception of methylene is uninterrupted or interrupted by oxygen or NR⁷, and
D and Y are as defined above.

Additionally, especially preferred reactive dyes of the invention are of the formula (1f)

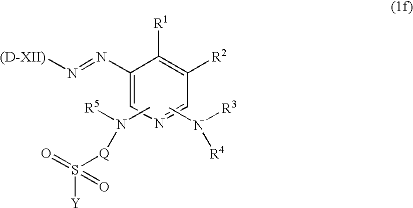

in which
R¹ is C₁-C₄-alkyl,
R² is cyano, carbamoyl or sulfomethyl,
R³ and R⁴ independently of one another are hydrogen or are C₁-C₁₈-alkyl or C₄-C₇-cycloalkyl each of which is unsubstituted or substituted by hydroxyl, sulfo, sulfato or vinylsulfonyl and with the exception of methyl is uninterrupted or interrupted by oxygen or NR⁷, in which R⁷ is hydrogen or C₁-C₄-alkyl, or R³ and R⁴ are part of heterocyclic groups,
R⁵ is hydrogen or C₁-C₁₈-alkyl,
Q is C₁-C₁₈-alkylene which is unsubstituted or substituted by C₁-C₄-alkyl, halogen or hydroxyl and with the exception of methylene is uninterrupted or interrupted by oxygen or NR⁷, and
(D-XII) and Y are as defined above.

The coupling components of the formula (2) in the dyes of the invention are new and likewise provided by the invention.

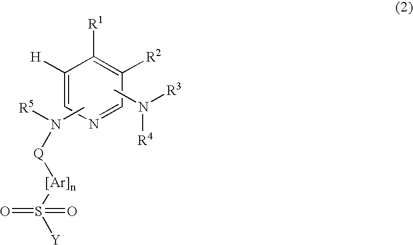

The present invention accordingly further provides diaminopyridines of the formula (2)

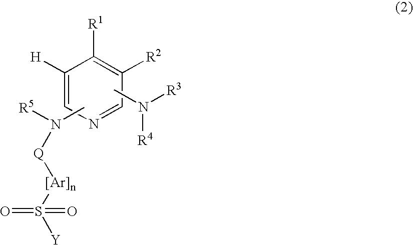

in which
R¹ is C₁-C₄-alkyl,
R² is cyano, carbamoyl or sulfomethyl,
R³, R⁴ and R⁵ independently of one another are hydrogen or are C₁-C₁₈-alkyl or C₄-C₇-cycloalkyl each of which is unsubstituted or substituted by hydroxyl, sulfo, sulfato or vinylsulfonyl and with the exception of methyl is uninterrupted or interrupted by oxygen or NR⁷, in which R⁷ is hydrogen or C₁-C₄-alkyl, or R³ and R⁴ are part of heterocyclic groups,
Q is C₁-C₁₈-alkylene which is unsubstituted or substituted by C₁-C₄-alkyl, halogen or hydroxyl and with the exception of methylene is uninterrupted or interrupted by oxygen or NR⁷,
n is 0 or 1,
Ar is a phenylene or naphthylene radical which is unsubstituted or substituted by sulfo, carboxyl, C₁-C₄-alkyl, C₁-C₄-alkoxy or halogen,
Y is —CH═CH₂ or —CH₂CH₂Y¹, in which
   Y¹ is hydroxyl or a group which can be eliminated under the action of alkali.

Examples of preferred diaminopyridines of the formula (2) are the diaminopyridines below, which can be in the form of an isomer mixture.

| Identification | Diaminopyridine |
|---|---|
| (P1) | 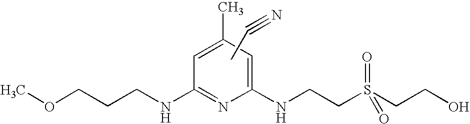 |
| (P2) | 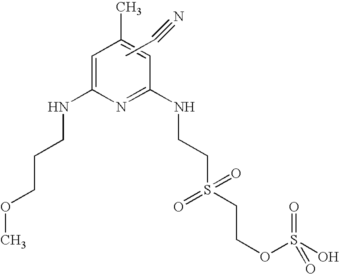 |
| (P3) | 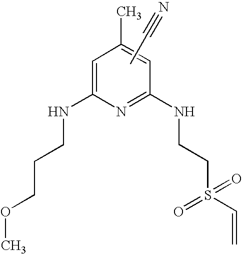 |
| (P4) | 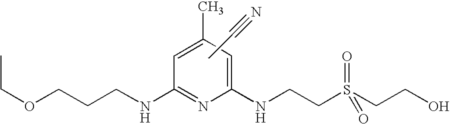 |
| (P5) | 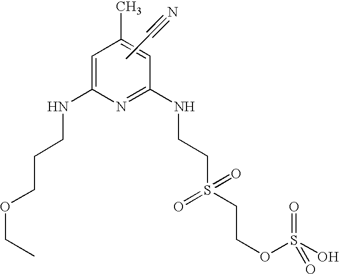 |
| (P6) | 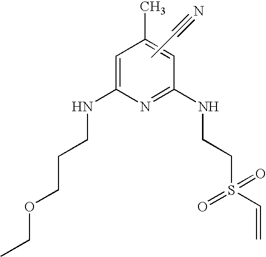 |
| (P7) | 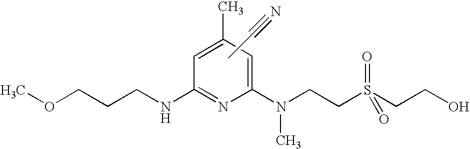 |
| (P8) | 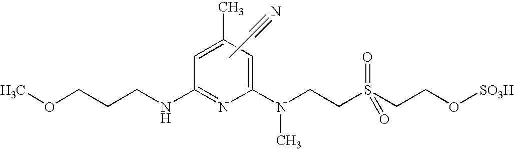 |
| (P9) | 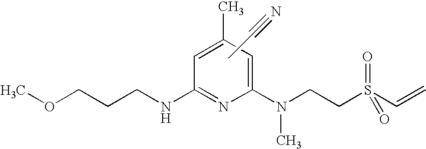 |
| (P10) | 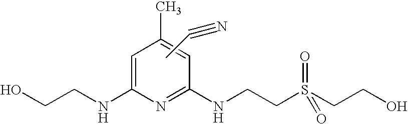 |
| (P11) | 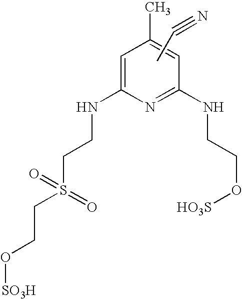 |
| (P12) | 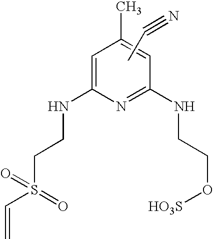 |
| (P13) | 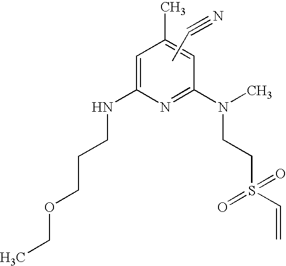 |
| (P14) | 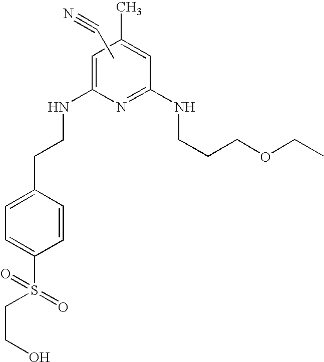 |
| (P15) | 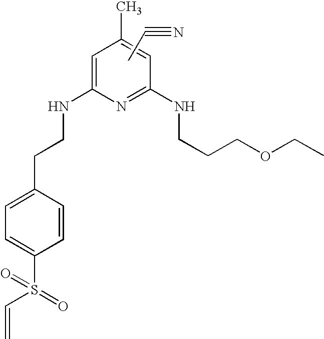 |
| (P16) | 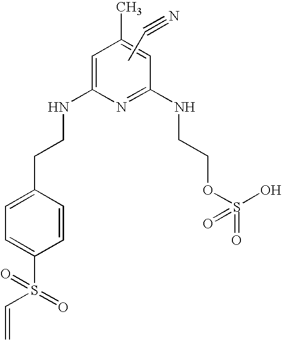 |
| (P17) | 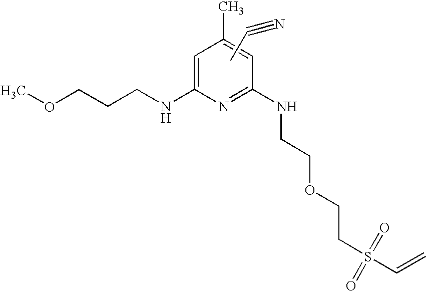 |
| (P18) | 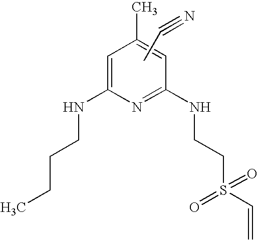 |
| (P19) | 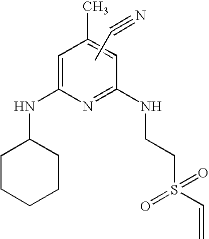 |
| (P20) | 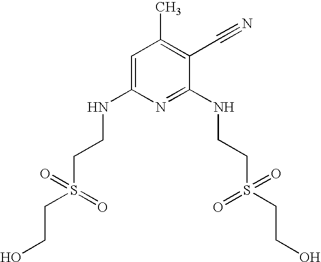 |
| (P21) | 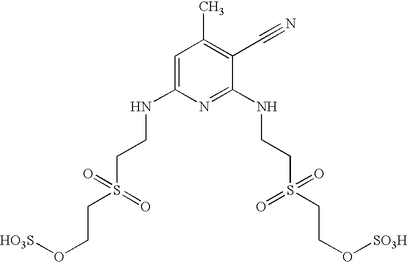 |
| (P22) | 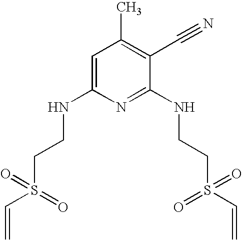 |
| (P23) | 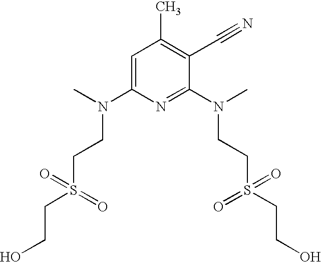 |
| (P24) | 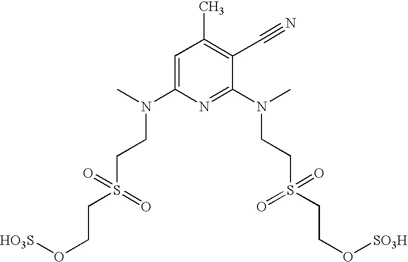 |
| (P25) | 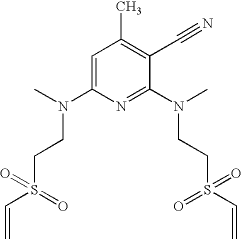 |

Where a compound of the formula (2) or of the formulae (P1) to (P25) contains an acid group, such as a group SO₃H, for example, it may also be present in the form of a salt, such as with the group SO₃M, for example, where M is hydrogen, an alkali metal, ammonium, one equivalent of an alkaline earth metal, or monovalent organic cations, especially alkylammonium.

The present invention further provides processes for preparing the diaminopyridines of the formula (2).

A first such process comprises reacting 2,6-dichloropyridine of the formula (3)

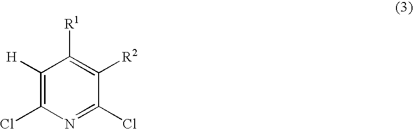

in which R¹ and R² are as defined above with one mole equivalent of an amine of the formula (4)

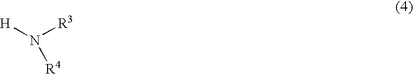

in which
R³ and R⁴ independently of one another are hydrogen or are C₁-C₁₈-alkyl or C₄-C₇-cycloalkyl each of which is unsubstituted or substituted by hydroxyl and/or sulfo and with the exception of methyl are uninterrupted or interrupted by oxygen, sulfone or sulfonyl (—SO₂—), or NR⁷, in which R⁷ is hydrogen or C₁-C₄-alkyl, or R³ and R⁴ are part of heterocyclic groups,
giving the monoaminopyridine of the formula (5) as an isomer mixture.

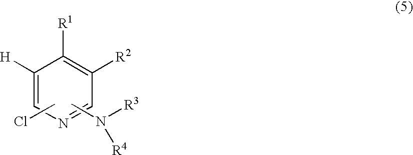

Alternatively it is possible first of all to prepare a compound of the formula (6)

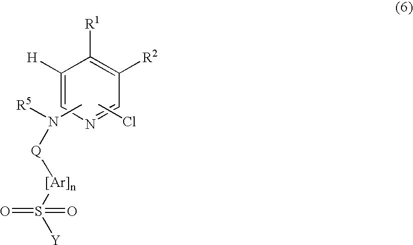

in which
R¹ is C₁-C₄-alkyl,
R² is cyano, carbamoyl or sulfomethyl,
R⁵ is hydrogen or is C₁-C₁₈-alkyl or C₄-C₇-cycloalkyl each of which is unsubstituted or substituted by hydroxyl and with the exception of methyl is uninterrupted or interrupted by oxygen or NR⁷, in which R⁷ is hydrogen or C₁-C₄-alkyl,
Q is C₁-C₁₈-alkylene which is unsubstituted or substituted by C₁-C₄-alkyl, halogen or hydroxyl and with the exception of methylene is uninterrupted or interrupted by oxygen or NR⁷,
n is 0 or 1,
Ar is a phenylene or naphthylene radical which is unsubstituted or substituted by sulfo, carboxyl, C₁-C₄-alkyl, C₁-C₄-alkoxy or halogen,
Y is CH₂CH₂OH,
   by
   a) reacting dichloropyridine of the formula (3) with one mole equivalent of an amine of the formula (7)

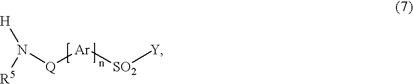

in which
Q, Ar, n and R⁵ are as defined above and

### Y is —CH₂CH₂OH, or

b) reacting dichloropyridine of the formula (3) with one mole equivalent of an amine of the formula (8)

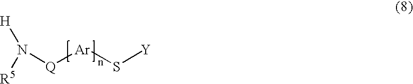

in which
R⁵ is hydrogen or is C₁-C₁₈-alkyl or C₄-C₇-cycloalkyl each of which is unsubstituted or substituted by hydroxyl and/or sulfo and with the exception of methyl is uninterrupted or interrupted by oxygen or NR⁷, in which R⁷ is hydrogen or C₁-C₄-alkyl,
Q is C₁-C₁₈-alkylene which is unsubstituted or substituted by C₁-C₄-alkyl, halogen or hydroxyl and with the exception of methylene is uninterrupted or interrupted by oxygen or NR⁷,
n is 0 or 1,
Ar is a phenylene or naphthylene radical which is unsubstituted or substituted by sulfo, carboxyl, C₁-C₄-alkyl, C₁-C₄-alkoxy or halogen, and
Y is —CH₂CH₂Y¹, in which
   Y¹ is hydroxyl,
      to form the corresponding monochloropyridine derivative of the formula (9)

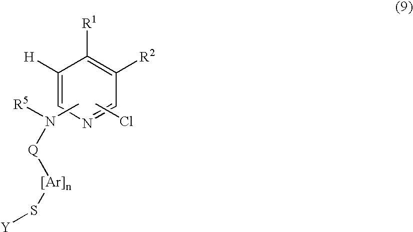

in which
R¹ is C₁-C₄-alkyl,
R² is cyano, carbamoyl or sulfomethyl,
R⁵ is hydrogen or is C₁-C₁₈-alkyl or C₄-C₇-cycloalkyl each of which is unsubstituted or substituted by hydroxyl and/or sulfo and with the exception of methyl is uninterrupted or interrupted by oxygen or NR⁷, in which R⁷ is hydrogen or C₁-C₄-alkyl,
Q is C₁-C₁₈-alkylene which is unsubstituted or substituted by C₁-C₄-alkyl, halogen or hydroxyl and with the exception of methylene is uninterrupted or interrupted by oxygen or NR⁷,
n is 0 or 1,
Ar is a phenylene or naphthylene radical which is unsubstituted or substituted by sulfo, carboxyl, C₁-C₄-alkyl, C₁-C₄-alkoxy or halogen, and
Y is —CH₂CH₂Y¹, in which
   Y¹ is hydroxyl
      followed by an oxidation of the compound (9) to form the monochloropyridine of the formula (6).

### Further Reaction of the Monochloropyridine

a) of the formula (6) with an amine of the formula (4) or
b) reaction of the monochloropyridine of the formula (5) with an amine of the formula (7)
or
c) reaction of the monochloropyridine of the formula (5) with an amine of the formula (8) and subsequent oxidation of the resultant intermediate of the formula (10)

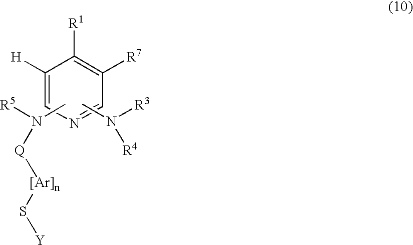

in which
R¹ is C₁-C₄-alkyl,
R² is cyano, carbamoyl or sulfomethyl,
R³ and R⁴ independently of one another are hydrogen or are C₁-C₁₈-alkyl or C₄-C₇-cycloalkyl each of which is unsubstituted or substituted by hydroxyl and/or sulfo and with the exception of methyl is uninterrupted or interrupted by oxygen, sulfone or sulfonyl (—SO₂—)ᵣ or NR⁷, in which R⁷ is hydrogen or C₁-C₄-alkyl, or R³ and R⁴ are part of heterocyclic groups,
R⁵ is hydrogen or is C₁-C₁₈-alkyl or C₄-C₇-cycloalkyl each of which is unsubstituted or substituted by hydroxyl and/or sulfo and with the exception of methyl is uninterrupted or interrupted by oxygen or NR⁷, in which R⁷ is hydrogen or C₁-C₄-alkyl,
Q is C₁-C₁₈-alkylene which is unsubstituted or substituted by C₁-C₄-alkyl, halogen or hydroxyl and with the exception of methylene is uninterrupted or interrupted by oxygen or NR⁷,
n is 0 or 1,
Ar is a phenylene or naphthylene radical which is unsubstituted or substituted by sulfo, carboxyl, C₁-C₄-alkyl, C₁-C₄-alkoxy or halogen,
Y is —CH₂CH₂Y¹, in which
   Y¹ is hydroxyl,
      or
      d) reaction of the monochloropyridine of the formula (9) with an amine of the formula (4) and subsequent oxidation of the resultant intermediate of the formula (10)
      gives a diaminopyridine of the formula (2), which is obtained as an isomer mixture, the ratio of the isomers being dependent on the synthesis route taken.

The oxidation takes place preferably in water, in an organic solvent or in a mixture of water and organic solvent. Examples of preferred organic solvents include acetic acid, dichloromethane and N-methylpyrrolidone. Preferred temperatures for said oxidation are temperatures from 10 to 80° C., in particular from 30 to 50° C. Said oxidation is carried out advantageously in the presence of a catalyst. Examples of such catalysts are salts with titanium, vanadium, manganese, molybdenum or tungsten, or clays and/or clay mineral earths. Particularly advantageous catalysts include, for example, sodium tungstate, montmorillonites and kaolin. Examples of suitable oxidizing agents are halogens or peroxides, such as chlorine, hydrogen peroxide, peroxodisulfates or peracids, such as m-chloroperbenzoic acid, peracetic acid or periodic acid and/or their salts, such as Oxone®, for example. In a strongly acidic medium said oxidation step can also be carried out using, for example, sulfur trioxide or oleum in the presence of catalysts containing iodine. A further example of the oxidation step is the reaction of a halogen, such as with chlorine, which may take place in a hydrochloric acid medium, for example.

The monoaminopyridines (5), (6) and (9) and the diaminopyridines (2) and (10) are prepared conventionally in water, in an organic solvent, such as toluene, butanol or N-methylpyrrolidone, for example, or without the use of a solvent, at temperatures from 30 to 150° C., preferably at 40 to 80° C., in the presence of a base, examples being alkali metal carbonate, alkali metal hydrogencarbonate, alkaline earth metal oxide, alkaline earth metal hydroxide, alkaline earth metal carbonate, or trialkylamine, preferably triethylamine or tributylamine.

Where appropriate it is possible to prepare the desired derivative by reaction with the corresponding acid derivative or corresponding acid, and then said derivative can be converted conventionally to the corresponding vinyl compound. The reaction of compounds of the formula (2) or compounds of the formula (10) in which
R¹ is C₁-C₄-alkyl,
R² is cyano, carbamoyl or sulfomethyl,
R³ and R⁴ independently of one another are hydrogen or are C₁-C₁₈-alkyl or C₄-C₇-cycloalkyl each of which is unsubstituted or substituted by hydroxyl and/or sulfo and with the exception of methyl is uninterrupted or interrupted by oxygen, sulfone or sulfonyl (—SO₂—), or NR⁷, in which R⁷ is hydrogen or C₁-C₄-alkyl, or R³ and R⁴ are part of heterocyclic groups,
R⁵ is hydrogen or is C₁-C₁₈-alkyl or C₄-C₇-cycloalkyl each of which is unsubstituted or substituted by hydroxyl and/or sulfo and with the exception of methyl is uninterrupted or interrupted by oxygen or NR⁷, in which R⁷ is hydrogen or C₁-C₄-alkyl,
Q is C₁-C₁₈-alkylene which is unsubstituted or substituted by C₁-C₄-alkyl, halogen or hydroxyl and with the exception of methylene is uninterrupted or interrupted by oxygen or NR⁷,
n is 0 or 1,
Ar is a phenylene or naphthylene radical which is unsubstituted or substituted by sulfo, carboxyl, C₁-C₄-alkyl, C₁-C₄-alkoxy or halogen,
Y is —CH₂CH₂Y¹, in which
   Y¹ is hydroxyl,
      with an acid derivative or an acid to give compounds of the formula (2) or, respectively, to give compounds of the formula (10) in which
R¹ is C₁-C₄-alkyl,
R² is cyano, carbamoyl or sulfomethyl,
R³ and R⁴ independently of one another are hydrogen or are C₁-C₁₈-alkyl or C₄-C₇-cycloalkyl each of which is unsubstituted or substituted by hydroxyl, sulfato or sulfo and with the exception of methyl is uninterrupted or interrupted by oxygen, sulfone or sulfonyl (—SO₂—), or NR⁷, in which R⁷ is hydrogen or C₁-C₄-alkyl, or R³ and R⁴ are part of heterocyclic groups,
R⁵ is hydrogen or is C₁-C₁₈-alkyl or C₄-C₇-cycloalkyl each of which is unsubstituted or substituted by hydroxyl, sulfato or sulfo and with the exception of methyl is uninterrupted or interrupted by oxygen or NR⁷, in which R⁷ is hydrogen or C₁-C₄-alkyl,
Q is C₁-C₁₈-alkylene which is unsubstituted or substituted by C₁-C₄-alkyl, halogen or hydroxyl and with the exception of methylene is uninterrupted or interrupted by oxygen or NR⁷,
n is 0 or 1,
Ar is a phenylene or naphthylene radical which is unsubstituted or substituted by sulfo, carboxyl, C₁-C₄-alkyl, C₁-C₄-alkoxy or halogen,
Y is —CH₂CH₂Y¹, in which
   Y¹ is a group which can be eliminated under the action of alkali takes place preferably in an organic solvent or without the use of solvents in the corresponding acid or acid derivative. Examples of preferred acids and acid derivatives are acetic anhydride, sulfuric acid, oleum and sulfur trioxide monohydrate. Preferred temperatures for the stated reaction are temperatures from −5 to 140° C., in particular from 5 to 40° C., The stated reaction with organic acid derivatives proceeds advantageously in the presence of a catalyst or water-remover, particularly preferred catalysts including pyridine or pyridine derivatives, such as 4-diaminopyridine, 4-pyrrolidinopyridine, nicotinic acid, or pyridonaphthyridine.

Alternatively the compound of the formula (10) can first be derivatized with an acid or acid derivative and then oxidized, in which case it is possible for oxidation to take place actually during the derivatization with an acid or acid derivative.

In the case of the described derivatizations with an acid or acid derivative it is also possible for substituents of R¹ to R⁵ or of the groups Q or Ar to be derivatized. For instance it is possible for esterifications, hydrolyses or oxidations, for example, to take place on the stated groups; for example, hydroxyl groups in R³ to R⁵ may be esterified likewise, as is also the case in the examples described later on below. Furthermore, in the course of the described oxidations, groups of R¹ to R⁵ or groups of Q or Ar or their substituents may also be derivatized.

The bis-reactive diaminopyridines of the formula (2) are likewise synthesized by stepwise introduction of the amino substituents, by reacting a dichloropyridine of the formula (3) with compounds of the formula (8), Y¹ being hydroxyl, followed by the above-described oxidation steps and followed where appropriate by the above-described derivatization steps, or by reacting a dichloropyridine of the formula (3) with compounds of the formula (7), Y¹ being hydroxyl, followed where appropriate by the above-described derivatization steps.

The dyes of the invention are accessible by reacting the corresponding diazonium salts with a coupling component of the formula (2) or (10), followed where appropriate by the oxidation and derivatization steps described above for the coupling component, in a similar way for the dye synthesis.

The diazonium salts are prepared conventionally by diazotization of the corresponding amines D-NH₂. Preferred amines for the diazotization are shown in table 1

TABLE 1

| Number | Amine |
|---|---|
| D-1 | 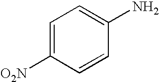 |
| D-2 | 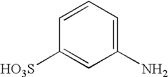 |
| D-3 | 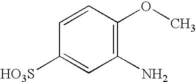 |
| D-4 | 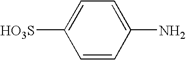 |
| D-5 | 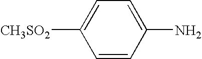 |
| D-6 | 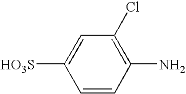 |
| D-7 | 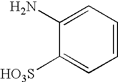 |
| D-8 | 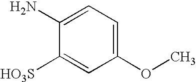 |
| D-9 | 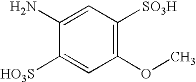 |
| D-10 | 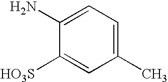 |
| D-11 | 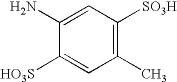 |
| D-12 | 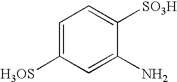 |
| D-13 | 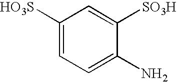 |
| D-14 | 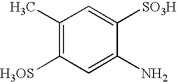 |
| D-15 | 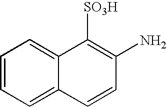 |
| D-16 | 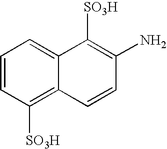 |
| D-17 | 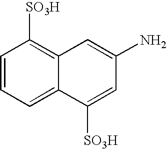 |
| D-18 | 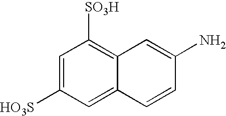 |
| D-19 | 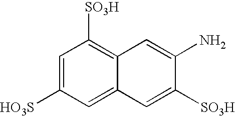 |
| D-20 | 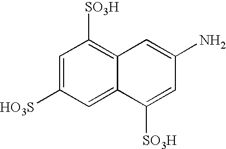 |
| D-21 | 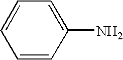 |
| D-22 | 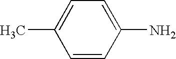 |
| D-23 | 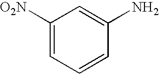 |
| D-24 | 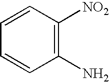 |
| D-25 | 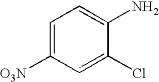 |
| D-26 | 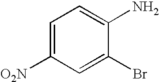 |
| D-27 | 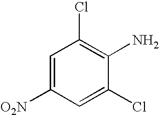 |
| D-28 | 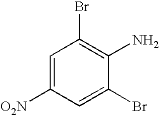 |
| D-29 | 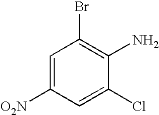 |
| D-30 | 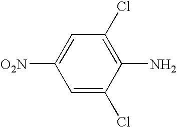 |
| D-31 | 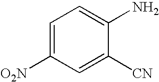 |
| D-32 | 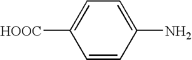 |
| D-33 | 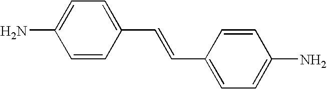 |
| D-34 | 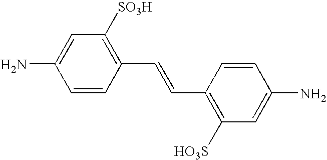 |
| D-35 | 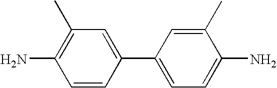 |
| D-36 | 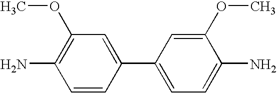 |
| D-37 | 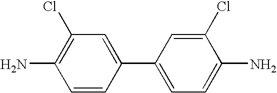 |
| D-38 | 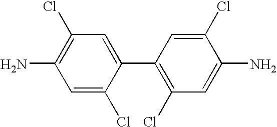 |
| D-39 | 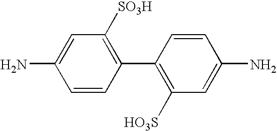 |
| D-40 | 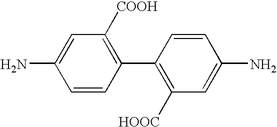 |
| D-41 | 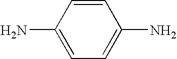 |
| D-42 | 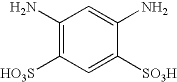 |
| D-43 | 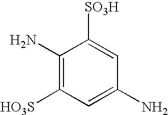 |
| D-44 | 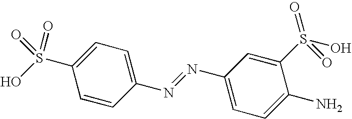 |
| D-45 | 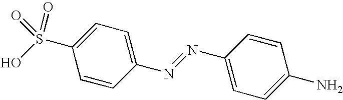 |
| D-46 | 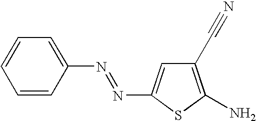 |
| D-47 | 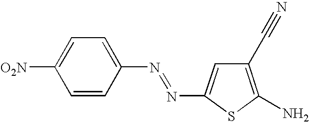 |
| D-48 | 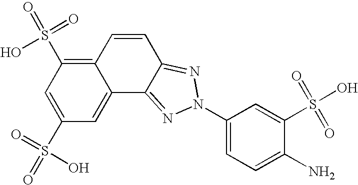 |
| D-49 | 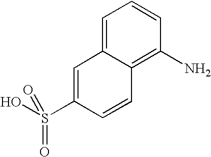 |
| D-50 | 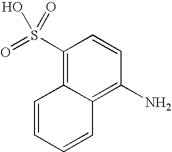 |
| D-51 | 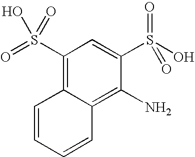 |
| D-52 | 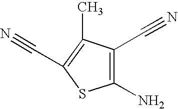 |
| D-53 | 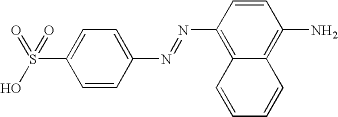 |
| D-54 | 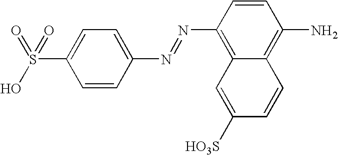 |
| D-55 | 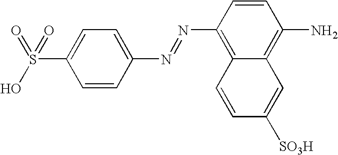 |
| D-56 | 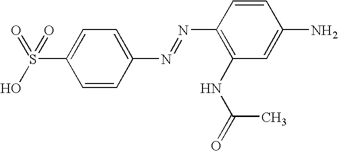 |
| D-57 | 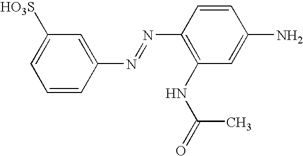 |
| D-58 | 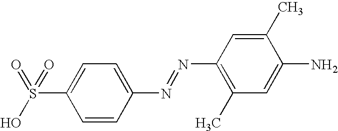 |
| D-59 | 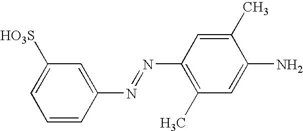 |
| D-60 | 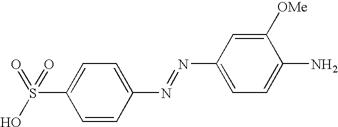 |
| D-61 | 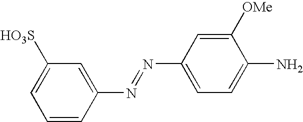 |
| D-62 | 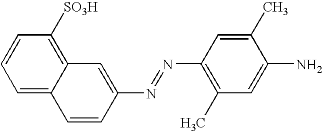 |
| D-63 | 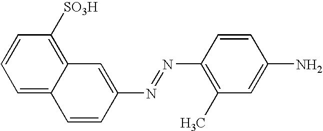 |
| D-64 | 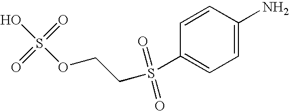 |
| D-65 | 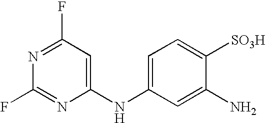 |
| D-66 | 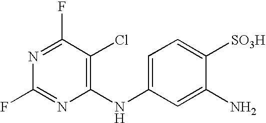 |
| D-67 | 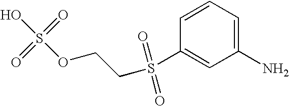 |
| D-68 | 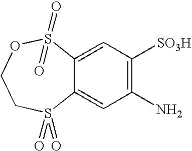 |
| D-69 | 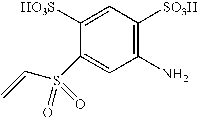 |
| D-70 | 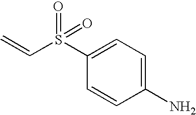 |
| D-71 | 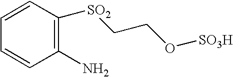 |
| D-72 | 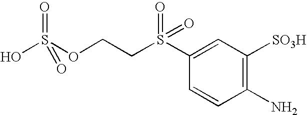 |
| D-73 | 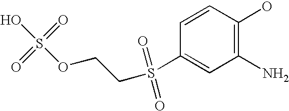 |
| D-74 | 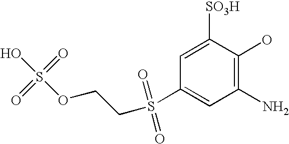 |
| D-75 | 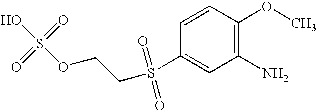 |
| D-76 | 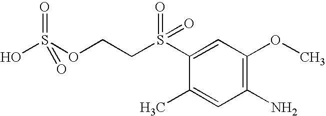 |
| D-77 | 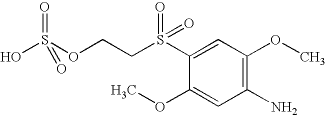 |
| D-78 | 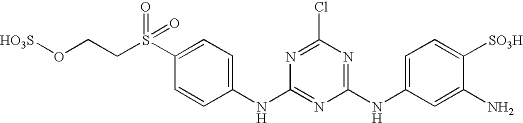 |
| D-79 | 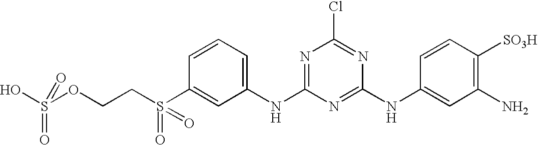 |
| D-80 | 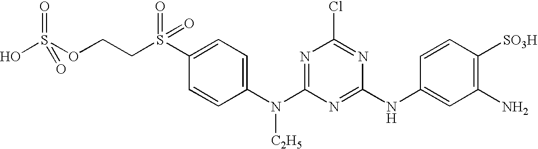 |
| D-81 | 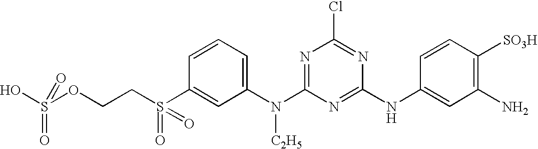 |
| D-82 | 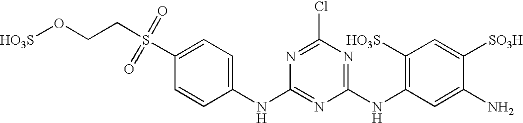 |
| D-83 | 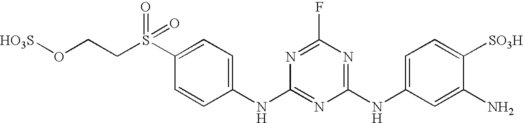 |
| D-84 | 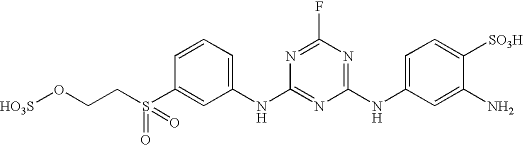 |
| D-85 | 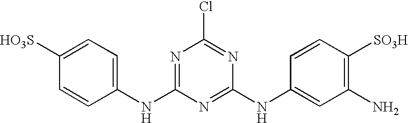 |
| D-86 | 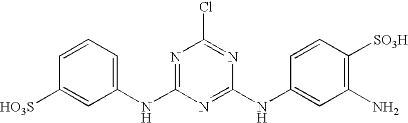 |
| D-87 | 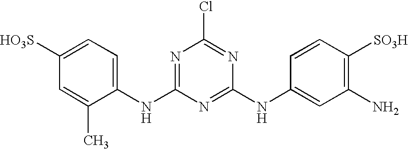 |
| D-88 | 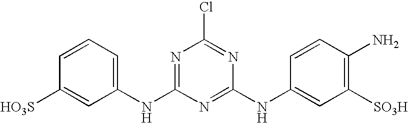 |
| D-89 | 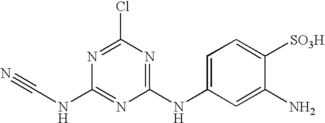 |
| D-90 | 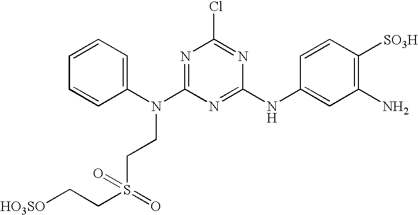 |
| D-91 | 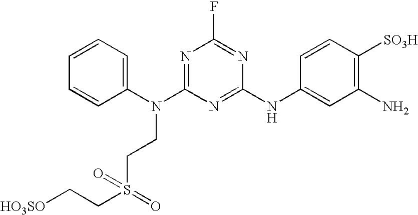 |
| D-92 | 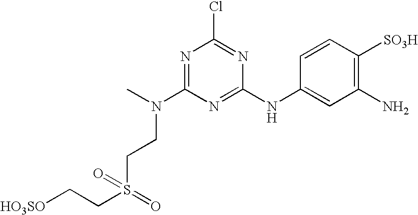 |
| D-93 | 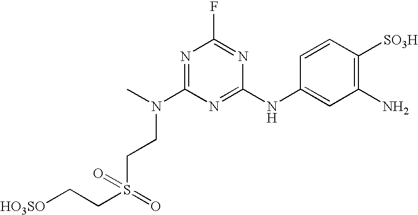 |
| D-94 | 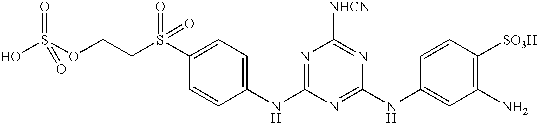 |
| D-95 | 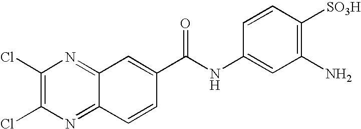 |
| D-96 | 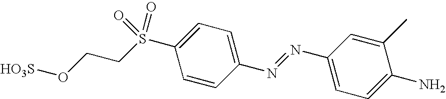 |
| D-98 | 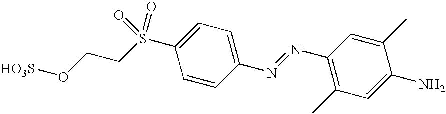 |
| D-99 | 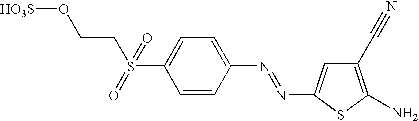 |
| D-100 | 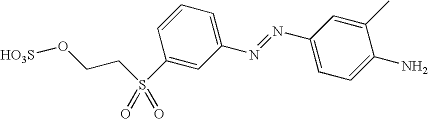 |
| D-101 | 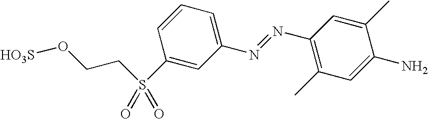 |
| D-102 | 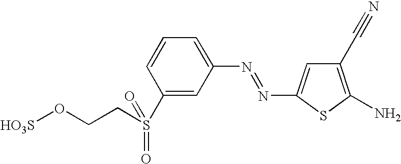 |

The dyes of the formula (1) of the invention are obtained as solutions or suspensions in the course of the preparation processes described above, and can be isolated by salting. They can also be spray-dried; evaporative concentration of the solution or suspension is also possible.

As a consequence of the synthesis, reactive dyes of the formula (1) of the invention are in the form of isomer mixtures, unless at one stage in the synthesis of the dye or diaminopyridine the isomers were separated or an isomerically pure coupler was used to synthesize the dye.

Furthermore, with the reactive dyes of the formula (1) of the invention, with the same chromophore, SO₂Y may firstly, preferably be β-sulfatoethylsulfonyl and secondly, more preferably, —SO₂CH═CH₂. The molar ratio of vinylsulfonyl dye to β-ethyl-substituted dye is preferably between 1:99 and 99:1.

The reactive dyes of the formula (1) of the invention are generally present as a formulation in solid or in liquid form. In solid form they generally include the electrolyte salts which are customary for water-soluble and, in particular, fiber-reactive dyes, such as sodium chloride, potassium chloride and sodium sulfate, and may further include the auxiliaries that are customary in commercial dyes, such as buffer substances capable of setting a pH between 3 and 7 in aqueous solution, such as sodium acetate, sodium borate, sodium hydrogencarbonate, sodium dihydrogenphosphate, sodium tricitrate and disodium hydrogenphosphate, small amounts of siccatives, or, if they are present in liquid form (including a proportion of thickeners of the kind customary in printing pastes), they may also include substances which ensure a long life for these formulations, such as mold preventatives, for example.

The reactive dyes of the formula (1) of the invention are preferably in the form of a dye powder or dye granules containing 10% to 80% by weight, based on the powder or granules, of an electrolyte salt, also referred to as a standardizing agent. Granules have particle sizes of, in particular, 50 to 500 μm. These solid formulations may also include the aforementioned buffer substances in a total amount of up to 10% by weight, based on the formulation. When the dyes are in a liquid formulation, the total dye content of these aqueous solutions is up to about 50%, such as between 5% and 50%, by weight, for example, the electrolyte salt content of these aqueous solutions being preferably below 10% by weight, based on the aqueous solution. The liquid formulations may include the aforementioned buffer substances in general in an amount of up to 10% by weight, preferably up to 2% by weight.

The reactive dyes of the formula (1) of the invention possess useful performance properties. They are used for dyeing and printing carboxamido- and/or hydroxyl- or amino-containing materials, in the form for example of sheetlike structures, such as paper and leather, or of films, as of polyamide, for example, or in the mass, such as polyamide and polyurethane, for example, but particularly in the form of fibers of the stated materials. They are used for dyeing and printing cellulosic fiber materials of all kinds. With preference they are also suitable for dyeing or printing polyamide fibers or blends of polyamide with cotton or with polyester fibers. It is also possible to use them to print textiles or paper by the ink-jet process.

The present invention accordingly also provides for the use of the reactive dyes of the formula (1) of the invention for dyeing or printing the stated materials, and provides methods of dyeing or printing such materials by conventional procedures, using one or more reactive dyes of the formula (1) of the invention as colorants.

Advantageously it is possible to make coloristic use of the as-synthesized solutions of the reactive dyes of the formula (1) of the invention, where appropriate following addition of a buffer substance and also, where appropriate, after concentration or dilution, as a liquid formulation, directly.

The stated materials are preferably employed in the form of fiber materials, particularly in the form of textile fibers, such as wovens or yarns, as in the form of hanks or wound packages.

Examples of carboxamido-containing materials are synthetic and natural polyamides and polyurethanes, especially in the form of fibers, examples being wool and other animal hairs, silk, leather, nylon-6,6, nylon-6, nylon-11 and nylon-4.

Hydroxyl-containing materials are those of natural or synthetic origin, examples being cellulose fiber materials or their regenerated products, and polyvinyl alcohols. Cellulose fiber materials are preferably cotton but also include other plant fibers, such as linen, hemp, jute and ramie fibers. Examples of regenerated cellulose fibers are staple viscose and filament viscose.

The reactive dyes of the formula (1) of the invention can be applied to and fixed on the stated substrates, particularly the stated fiber materials, by the application techniques that are known for water-soluble dyes, in particular for fiber-reactive dyes.

Additionally, wool which has been given a nonfelting or low-felting finish (cf., for example, H. Rath, Lehrbuch der Textilchemie, Springer-Verlag, 3rd edition (1972), pp. 295-299, especially finishing by the Hercosett process (p. 298); J. Soc. Dyers and Colourists 1972, 93-99′ and 1975, 33-44) can be dyed with very good fastness properties. The process of dyeing on wool is accomplished here in conventional manner from an acidic medium. For instance, acetic acid and/or ammonium sulfate or acetic acid and ammonium acetate or sodium acetate can be added to the dyebath to obtain the desired pH. To obtain a dyeing of acceptable levelness, it is advisable to add customary leveling assistants, based for example on a reaction product of cyanuric chloride with three times the molar amount of an aminobenzenesulfonic acid and/or an aminonaphthalenesulfonic acid, or based on a reaction product of, say, stearylamine with ethylene oxide. For example, the dye mixture of the invention is preferably subjected to the exhaust process initially from an acidic dyebath having a pH of about 3.5 to 5.5, under pH monitoring, and then the pH, toward the end of the dyeing time, is shifted into the neutral and optionally weakly alkaline range up to a pH of 8.5, in order to bring about—especially for very deep dyeings—the full reactive bond between the dyes of the dye mixtures of the invention and the fiber. At the same time the non-reactive dye fraction is removed. The procedure described here also applies to the production of dyeings on fiber materials made from other natural polyamides or from synthetic polyamides and polyurethanes. These materials can be dyed using the customary dyeing and printing processes, which are described in the literature and known to the skilled worker (see, for example, H.-K. Rouette, Handbuch der Textilveredlung, Deutscher Fachverlag GmbH, Frankfurt am Main). Besides including the dyes of the formulae (I) and water, the dyeing liquors and printing pastes may also include further additives. Examples of additives are wetting agents, antifoams, leveling agents, and agents which influence the properties of the textile material, such as softeners, flame retardants, soil, water and oil repellents or water softeners. Printing pastes in particular may also include natural or synthetic thickeners, such as alginates and cellulose ethers, for example. In the dyebaths and printing pastes the amounts of dye may vary within wide limits in accordance with the desired depth of shade. Generally speaking, the amounts of the dyes of the formula (1) are from 0.01% to 15% by weight, in particular from 0.1% to 10% by weight, based on the material to be dyed and on the printing paste, respectively.

On cellulose fibers, dyeing from a long liquor by the exhaust process and using any of a very wide variety of acid binders and, where appropriate, neutral salts, such as sodium chloride or sodium sulfate, dyeings are obtained that have very good color yields. In the case of the exhaust process it is preferred to carry out dyeing at a pH of 3 to 7, in particular at a pH of 4 to 6. The liquor ratio can be selected from within a wide range and is for example between 3:1 and 50:1, preferably between 5:1 and 30:1. In an aqueous bath preference is given to dyeing at temperatures between 40 and 105° C., where appropriate at a temperature up to 130° C. under superatmospheric pressure, and in the presence, where appropriate, of customary dyehouse assistance. To enhance the wetfasteness properties of the dyed material, an aftertreatment can be carried out to remove unfixed dye. This aftertreatment takes place in particular at a pH of 8 to 9 and at temperatures from 75 to 80° C.

One possible procedure here is to introduce the material into the hot bath, to raise the bath temperature gradually to the desired level, and to complete the dyeing operation. The neutral salts which accelerate the exhaustion of the dyes can if desired not be added to the bath until after the actual dyeing temperature has been reached.

By the padding process as well, excellent color yields and a very good build-up of color are obtained on cellulose fibers, and the dyeings can be fixed conventionally by batching at room temperature or elevated temperature, at up to about 60° C., for example, by steam treatment or by means of dry heat.

Similarly, the customary printing processes for cellulose fibers, which can be carried out in one phase—for example, by printing with a printing paste containing sodium bicarbonate or another acid binder, followed by steam treatment at 100 to 103° C.—or in two phases—for example, by printing with neutral or weakly acidic printing ink and then fixing, either by passage through a hot, electrolyte-containing alkaline bath or by overpadding with an alkaline, electrolyte-containing padding liquor and subsequent batching or steam treatment or dry heat treatment of the alkali-overpadded material—produce strongly colored prints with high contour definition and a clear white ground. Print outcome is not strongly tied to varying fixing conditions.

In the case of fixing by means of dry heat in accordance with the conventional thermofixing methods, hot air at 120 to 200° C. is used. As well as the usual steam at 101 to 103° C., it is also possible to employ superheated steam and pressurized steam with temperatures up to 160° C.

The acid binders and agents which effectuate the fixing of the dyes on the cellulose fibers are, for example, water-soluble basic salts of the alkali metals and likewise alkaline earth metals with organic or inorganic acids, or compounds which release alkali under heating. Mention may be made in particular of the alkali metal hydroxides and alkali metal salts of weak to moderately strong organic or inorganic acids, preference among the alkali metal compounds being intended for the sodium and potassium compounds. Examples of such acid binders are sodium hydroxide, potassium hydroxide, sodium carbonate, sodium bicarbonate, potassium carbonate, sodium formate, sodium dihydrogenphosphate, disodium hydrogenphosphate, sodium trichloroacetater waterglass or trisodium phosphate.

The reactive dyes of the formula (1) of the invention are notable for high reactivity, high fixing capacity, very high build-up capacity, and high lightfastness, including perspiration lightfastness. They can therefore be employed by the exhaust dyeing process at low dyeing temperatures, and in the case of pad-steam processes require only short steaming times. The degrees of fixing are high, and the portions not fixed can easily be washed off, the difference between degree of exhaustion and degree of fixing being remarkably small—that is, the soaping loss is very low. They are also particularly suitable for printing, especially over cotton, but also for printing nitrogen-containing fibers, such as wool or silk, or blends containing wool or silk.

The reactive dyes of the formula (1) of the invention are notable for the fact that, following the dyeing operation, unfixed dye portions on the fiber material are readily washed off without the dye which becomes detached tinting adjacent whites in the wash. This produces advantages for the dyeing operation, by saving on wash cycles and hence costs.

The dyeings and prints produced with the dyes of the formula (1) of the invention, particularly on polyamides, exhibit high color strength and high fiber— dye bond stability not only in the acidic but also in the alkaline range, and also good lightfastness and very good wetfastness properties, such as washing, water, seawater, cross-dyeing and perspiration fastnesses, and also good fastness to dry heat setting, to pleating and to crocking.

The present invention further provides for the use of the above-mentioned dyes of the formula (1) in printing inks for digital textile printing by the ink-jet process.

The printing inks of the invention comprise one or more of the stated reactive dyes, in amounts for example of 0.1% to 50% by weight, preferably in amounts of 1% to 30% by weight, and more preferably in amounts of 1% to 15% by weight, based on the total ink weight. Likewise included may be combinations of the stated reactive dyes with other reactive dyes used in textile printing. For the inks to be used in a continuous flow process, a conductivity of 0.5 to 25 mS/m can be set by adding electrolyte.

Examples of suitable electrolyte include the following: lithium nitrate, potassium nitrate.

The dye inks of the invention may contain organic solvents in a total amount of 1-50%, preferably of 5-30% by weight.

Examples of suitable organic solvents are

alcohols, for example, methanol, ethanol, 1-propanol, isopropanol, 1-butanol, tert-butanol, pentyl alcohol, polyhydric alcohols, for example, 1,2-ethanediol, 1,2,3-propanetriol, butanediol, 1,3-butanediol, 1,4-butanediol, 1,2-propanediol, 2,3-propanediol, pentanediol, 1,4-pentanediol, 1,5-pentanediol, hexanediol, D,L-1,2-hexanediol, 1,6-hexanediol, 1,2,6-hexanetriol, 1,2-octanediol, polyalkylene glycols, for example, polyethylene glycol, polypropylene glycol, alkylene glycols having 2 to 8 alkylene groups, for example, monoethylene glycol, diethylene glycol, triethylene glycol, tetraethylene glycol, thioglycol, thiodiglycol, butyl triglycol, hexylene glycol, propylene glycol, dipropylene glycol, tripropylene glycol, lower alkyl ethers of polyhydric alcohols, for example, ethylene glycol monomethyl ether, ethylene glycol monoethyl ether, ethylene 1 glycol monobutyl ether, diethylene glycol monomethyl ether, diethylene glycol monoethyl ether, diethylene glycol monobutyl ether, diethylene glycol monohexyl ether, triethylene glycol monomethyl ether, triethylene glycol monobutyl ether, tripropylene glycol monomethyl ether, tetraethylene glycol monomethyl ether, tetraethylene glycol monobutyl ether, tetraethylene glycol dimethyl ether, propylene glycol monomethyl ether, propylene glycol monoethyl ether, propylene glycol monobutyl ether, tripropylene glycol isopropyl ether, polyalkylene glycol ethers, for example, polyethylene glycol monomethyl ether, polypropylene glycol glycerol ether, polyethylene glycol tridecyl ether, polyethylene glycol nonylphenyl ether,
amines, for example, methylamine, ethylamine, triethylamine, diethylamine, dimethylamine, trimethylamine, dibutylamine, diethanolamine, triethanolamine, N-formylethanolamine, ethylenediamine,
urea derivatives, for example, urea, thiourea, N-methylurea, N,N′-epsilon-dimethylurea, ethyleneurea, 1,1,3,3-tetramethylurea, N-acetylethanolamine, amides, for example, dimethylformamide, dimethylacetamide, acetamide, ketones or keto alcohols, for example, acetone, diacetone alcohol, cyclic ethers, for example, tetrahydrofuran, trimethylolethane, trimethylolpropane, 2-butoxyethanol, benzyl alcohol, 2-butoxyethanol, gamma-butyrolactone, epsilon-caprolactam, additionally sulfolane, dimethylsulfolane, methylsulfolane, 2,4-dimethylsulfolane, dimethyl sulfone, butadiene sulfone, dimethyl sulfoxide, dibutyl sulfoxide, N-cyclohexylpyrrolidone, N-methyl-2-pyrrolidone, N-ethylpyrrolidone, 2-pyrrolidone, 1-(2-hydroxyethyl)-2-pyrrolidone, 1-(3-hydroxypropyl)-2-pyrrolidone, 1,3-dimethyl-2-imidazolidinone, 1,3-dimethyl-2-imidazolinone, 1,3-bismethoxymethylimidazolidine, 2-(2-methoxyethoxy)ethanol, 2-(2-ethoxyethoxy)ethanol, 2-(2-butoxyethoxy)ethanol, 2-(2-propoxyethoxy)ethanol, pyridine, piperidine, butyrolactone, trimethylolpropaner 1,2-dimethoxypropane, dioxane, ethyl acetate, ethylenediaminetetraacetate, ethyl pentyl ether, 1,2-dimethoxypropane, trimethylpropane.

The printing inks of the invention may further comprise the customary additives, such as viscosity moderators to set viscosities in the range from 1.5 to 40.0 mPas in a temperature range from 20 to 50° C. Preferred inks have a viscosity of 1.5 to 20 mPas, particularly preferred inks a viscosity of 1.5 to 15 mPas.

Suitable viscosity moderators include rheological additives, examples of which include the following:

polyvinylcaprolactam, polyvinylpyrrolidone, and their copolymers, polyetherpolyol, associative thickeners, polyurea, polyurethane, sodium alginates, modified galactomannans, polyetherurea, polyurethane, nonionic cellulose ethers.

As further additives the inks of the invention may include surface-active substances for setting surface tensions of 20 to 65 mN/m, which where appropriate are adapted to the technique used (thermo or piezo technology). Examples of suitable surface-active substances include the following: surfactants of all kinds, preferably nonionic surfactants, butyldiglycol, 1,2-hexanediol.

The inks may further comprise customary additives, such as fungal and bacterial growth inhibitors in amounts of 0.01% to 1% by weight, based on the total ink weight.

The inks may be prepared in conventional manner by mixing of the components in water.

The dye inks of the invention are suitable for use in inkjet printing processes for printing any of a very wide variety of pretreated materials, such as silk, leather, wool, cellulosic fiber materials of all kinds, and polyurethanes, and especially polyamide fibers. The printing inks of the invention are also suitable for printing pretreated hydroxyl-containing and/or amino-containing fibers present in blends, examples being blends of cotton, silk, wool with polyester fibers or polaymide fibers.

In contrast to conventional textile printing, where the printing ink already includes all of the fixing chemicals and thickeners for a reactive dye, in the case of ink-jet printing the auxiliaries must be applied to the textile substrate in a separate pretreatment step.

Pretreatment of the textile substrate, such as cellulose and regenerated cellulose fibers and also silk and wool, is carried out with an aqueous alkaline liquor prior to printing. To fix reactive dyes alkali is needed, examples being sodium carbonate, sodium bicarbonate, sodium acetate, trisodium phosphate, sodium silicate, sodium hydroxide, alkali donors such as, for example, sodium chloroacetate, sodium formate, hydrotropic substances such as, for example, urea, reduction inhibitors, such as, for example, sodium nitrobenzenesulfonates, and also thickeners to prevent flowing of the motifs when the printing ink is applied—examples of these are sodium alginates, modified polyacrylates and highly etherified galactomannans.

These pretreatment reagents are applied uniformly to the textile substrate in a defined amount using suitable applicators, such as with a 2- or 3-roll pad, with contactless spraying technologies, by means of foam application, or using appropriately adapted ink-jet technologies, and the treated substrate is subsequently dried.

After printing, the textile fiber material is dried at 120 to 150° C. and then fixed. The ink-jet prints produced with reactive dyes can be fixed at room temperature or with saturated steam, with superheated steam, with hot air, with microwaves, with infrared radiation, with laser or electron beams or with other suitable energy transfer methods.

A distinction is made between one- and two-phase fixing operations:

In one-phase fixing, the chemicals needed for fixing are already on the textile substrate.

In two-phase fixing this pretreatment is unnecessary. Fixing requires only alkali, which, following ink-jet printing and prior to the fixing operation, is applied without intermediate drying. There is no need for further additives such as urea or thickener.

Fixing is followed by print aftertreatment, which is the prerequisite for good fastnesses, high brilliance and an impeccable white ground.

The prints produced with the dye inks of the invention, particularly on polyamide, possess high color strength and high fiber-dye bond stability in not only the acidic but also the alkaline range, and additionally possess good lightfastness and very good wetfastness properties, such as wash, water, seawater, cross-dyeing and perspiration fastnesses, and also good fastness to dry heat setting and pleating and to crocking.

The examples below serve to illustrate the invention. Parts and percentages are by weight unless noted otherwise. The relationship between parts by weight and parts by volume is that of the kilogram to the liter.

The compounds described in terms of formulae in the examples are given in the form of the free acid. Generally, however, they are prepared and isolated in the form of their alkali metal salts, such as lithium, sodium or potassium salts, and used for dyeing in the form of their salts. Similarly, the starting compounds and components indicated in the form of the free acid in the examples below, especially in tabular examples, can be used as such in the synthesis or in the form of their salts, preferably alkali metal salts.

### EXAMPLE 1

Introduced into a mixture of 400 ml of toluene and 100 ml of triethylamine are so 187 g of 2,6-dichloro-4-methylnicotinonitrile (3-cyano-2,6-dichloro-4-methylpyridine) and 89 g of 3-methoxypropylamine. The mixture is heated to 50° C. and stirred at that temperature for 16 hours.

The reaction mixture is cooled to 0° C. The crystals obtained are filtered off with suction, washed with ether, dried and stirred into 1.5 l of water, adjusted to a pH of 1 using hydrochloric acid, filtered off again with suction, washed to neutrality with water, filtered off once more with suction, and dried. This gives only one isomer of the formula (5-1)

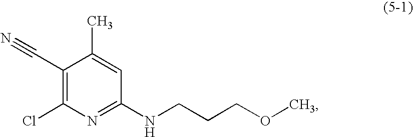

the other isomer remaining in solution. Into 50 ml of 2-(2-aminoethylsulfanyi)ethanol 12 g of the resulting product are introduced, and the mixture is heated to 110° C. After 4 hours the reaction mixture is allowed to cool and is poured onto 200 g of ice, the solid product is filtered off with suction and washed with water, and the filtercake is dried. This gives the product of the formula (10-1)

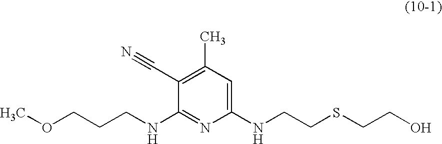

The resultant diaminopyridine of the formula (10-1) is introduced into 100 ml of water together with 0.165 g of sodium tungstate dihydrate in 1 ml of ethanol and the mixture is heated to 50° C. Added to this mixture over the course of 6 hours are 15 ml of 35% strength hydrogen peroxide solution. This gives the diaminopyridine of the formula (2-1). It is isolated, after cooling, by suction filtration.

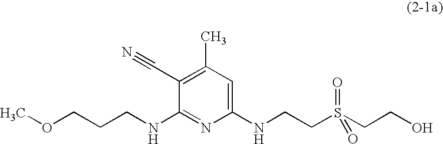

The diaminopyridine of the formula (2-1a) is stirred together with methanol and added to a reaction mixture of a diazonium salt obtained by conventionally diazotizing 8.9 g of 2-amino-5-(4-sulfophenylazo)benzensulfonic acid (D-44). Using sodium carbonate solution, the pH of the mixture is held at 3.5 to 4.5 for about 2 hours and then set at 6.5. The solid product is obtained conventionally by salting out with sodium chloride and suction filtration, and the resultant presscake is dried. The product of the formula (1-1a) is obtained in the form of the disodium salt, written in the form of the free acid—thus:

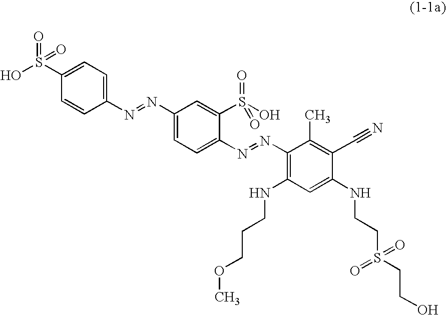

10 g of the resultant product are introduced at a temperature of 10-15° C. into 30 ml of 100% sulfuric acid (monohydrate) and the mixture is stirred at this temperature for 30 minutes. The reaction mixture is discharged onto 200 g of ice and at a temperature of 0-5° C. it is adjusted to a pH of 6 using 50 g of calcium carbonate, and then filtered. Evaporation of the filtrate gives dye (1-1b) in the form of the trisodium salt, written—in the form of the free acid—thus:

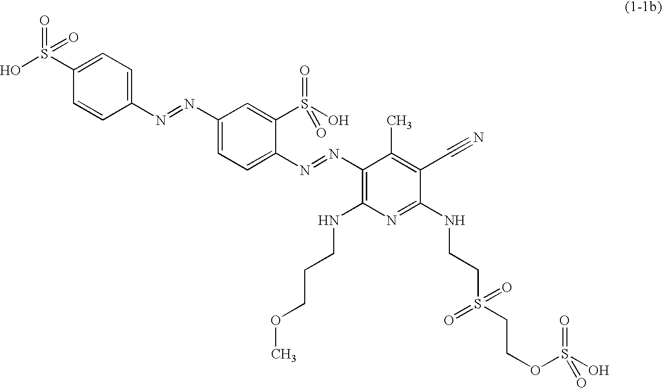

which dyes polyamide in red shades. The dyeings are notable for good all-round fastness properties, particularly light and wet fastnesses.

Alternatively the filtrate is adjusted to a pH of 10 to 11 using 20% strength sodium hydroxide solution, stirred at this pH for 30 minutes, adjusted to a pH of 6 to 6.5 using hydrochloric acid, and evaporated. The residue contains the product of (1-1c) in the form of the disodium salt, written—in the form of the free acid thus:

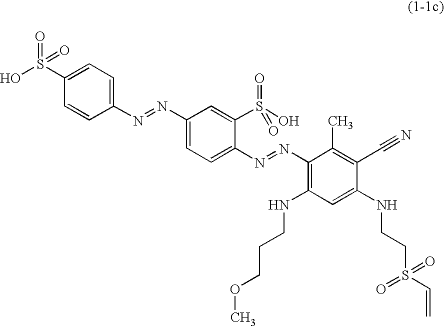

which dyes polyamide in red shades. The dyeings are notable for good all-round fastnesses, particularly light and wet fastnesses.

### EXAMPLE 2

Into 100 ml of N-methylpyrrolidone there are introduced 12 g of the product of the formula (5-1) obtained by example 1, 15 ml of 15% strength sodium carbonate solution and 13 g of 2-(2-methylaminoethylsulfanyi)ethanol. The mixture is heated at 140° C. until reaction is complete.

The reaction mixture contains the product of the formula (10-2).

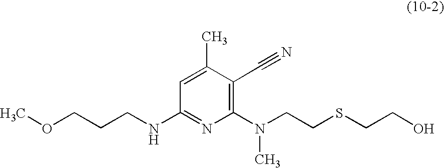

The reaction mixture obtained is admixed with 100 ml of water and 0.165 g of sodium tungstate dihydrate and is heated to 50° C., at which temperature 19 ml of a 35% strength hydrogen peroxide solution is added dropwise over approximately 2 hours, after which the reaction mixture is heated to 70 to 75° C. and stirred at this temperature for 3 hours. It contains the product of the formula (2-2a).

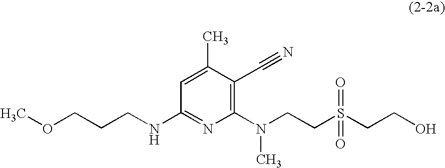

The reaction mixture can be used without further workup for coupling with diazonium salts.

To isolate the product, 20% strength sodium chloride solution is added and the precipitated solid is filtered off with suction, washed and dried.

10 g of the resultant product of the formula (2-2a) are introduced at a temperature of 10-15° C. into 30 ml of 100% sulfuric acid (monohydrate) and the mixture is stirred at this temperature for 5 hours. The reaction mixture is discharged onto 200 g of ice, adjusted to a pH of 6 using about 60 g of calcium carbonate at a temperature of 0-5° C., and filtered.

The filtrate contains the compound of the formula (2-2b) in the form of the sodium salt, written—in the form of the free acid—thus:

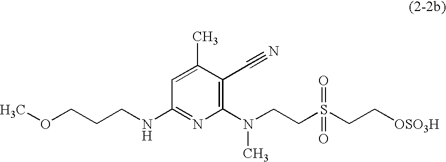

The resultant solution (the filtrate) is adjusted to a pH of 10 to 11 using 20% strength sodium hydroxide solution, stirred at this pH for 60 minutes and adjusted to a pH of 6 to 6.5 using hydrochloric acid, and the precipitated solid is filtered off with suction, washed and dried. This gives a product containing a compound of the formula (2-2c).

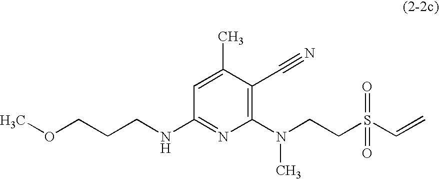

Preparation of the Dye Starting from (2-2a):

The product of the formula (2-2a) is added at a temperature of 15 to 20° C. to a reaction mixture of a diazonium salt obtained by conventionally diazotizing 12.5 g of 2-amino-5-(4-sulfophenylazo)benzenesulfonic acid (D-44).

Using sodium carbonate solution, the pH of the mixture is held at 1.5 for about 2 hours and then set at 6. The mixture is heated to 35° C. and then the product is isolated by salting out with sodium thiocyanate and suction filtration. The presscake obtained is digested with acetone until thiocyanate is no longer detectable in the acetone, and then is dried. This gives a product of the formula (1-2a) in the form of the disodium salt, written—in the form of the free acid—thus:

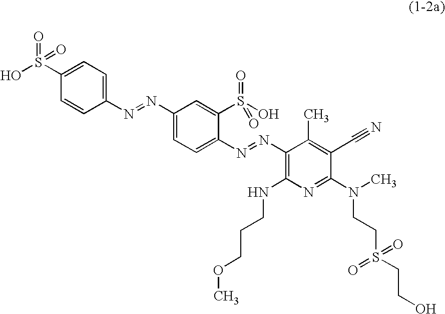

The resulting product is introduced at a temperature of 10-15° C. into 45 ml of 100% sulfuric acid (monohydrate) and the mixture is stirred at this temperature for 16 hours. The reaction mixture is discharged onto 300 g of ice, adjusted to a pH of 6 at a temperature of 0-5° C. using 85 g of calcium carbonate, and filtered. The filtrate contains the product of the formula (1-2b) in the form of the trisodium salt, written—in the form of the free acid—thus:

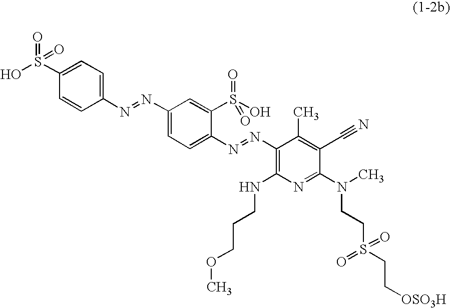

which dyes polyamide in red shades. The dyeings are notable for good all-round fastnesses, particularly light and wet fastnesses.

The resulting filtrate is adjusted to a pH of 10 to 11 using 20% strength sodium hydroxide solution, stirred at this pH for one hour, adjusted to a pH of 6 to 6.5 using hydrochloric acid, and concentrated by evaporation. This gives the product of the formula (1-2c) in the form of the disodium salt, written—in the form of the free acid—thus:

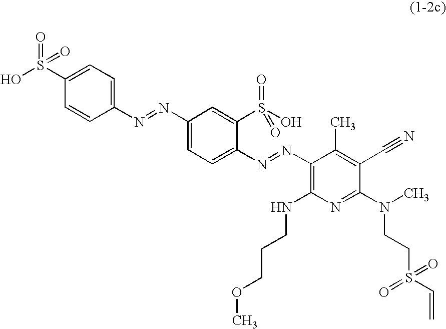

which dyes polyamide in red shades. The dyeings are notable for good all-round fastnesses, particularly light and wet fastnesses.

The dye of the formula (1-2c) can also be obtained by coupling the diazonium salt of the di-yellow acid (D44) onto the coupler of the formula (2-2c).

### EXAMPLE 3

Into 400 ml of toluene there are introduced at 75° C. 187 g of 2,6-dichloro-4-methylnicatinonitrile (3-cyano-2,6-dichloro-4-methylpyridine) and 121 g of 2-(2-aminoethylsulfanyl)ethanol 101 g of triethylamine are added dropwise, the mixture is stirred at 75° C. for 24 hours and filtered, the toluene phase is washed with water and then separated off, and the solvent is evaporated under reduced pressure. This gives a product mixture of the following compounds (formula 9-3a):

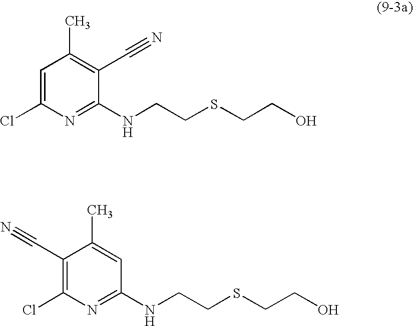

Half of the resultant product is dissolved in a mixture of 500 ml of N-methylpyrrolidone, 250 ml of water and 1.65 g of sodium tungstate dehydrate. The mixture is heated to 30° C. and 200 ml of 30% strength hydrogen peroxide solution are added dropwise, in the course of which the temperature is permitted to rise to 75° C. The product mixture obtained corresponds to the following formulae (6-3):

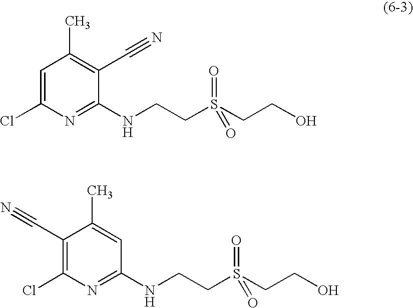

The reaction mixture is admixed with 62 g of ethanolamine (2-aminoethanol) and heated to 105-120° C. At the end of the reaction the reaction mixture (820 g of solution A) contains the following compounds (2-3a):

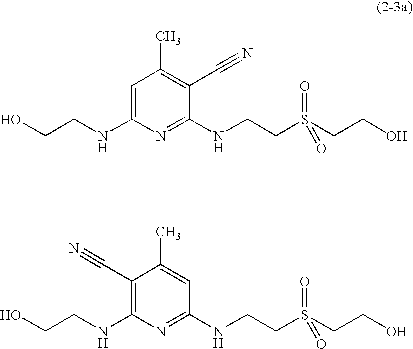

The product (2-3a) can also be obtained as follows:

Half of the product of example 3, formula (9-3) is introduced into 62 g of ethanolamine (2-aminoethanol) and the mixture is heated to 105-120° C. At the end of the reaction the reaction mixture contains the following compounds (formula 10-3):

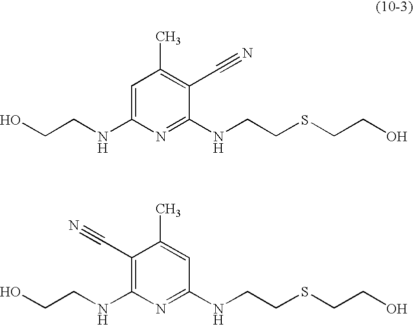

The resultant reaction mixture is admixed with 50 ml of water and 100 ml of N-methylpyrrolidone and heated to 50° C., 1.65 g of sodium tungstate dihydrate are added to the mixture, 240 ml of 30% strength hydrogen peroxide solution are added dropwise at a rate such that the temperature does not rise above 75° C., and the reaction mixture is stirred at this temperature until reaction is complete—approximately 5 hours. At the end of the reaction the reaction mixture (406 g of solution B) contains the compounds (2-3a). The reaction mixture can be used without further workup for coupling with diazonium salts. To isolate the product, 20% strength sodium chloride solution is added and the precipitated solid is filtered off with suction, washed and dried.

10 g of the resultant product are introduced at a temperature of 10-15° C. into 30 ml of 100% sulfuric acid (monohydrate) and the mixture is stirred at this temperature for 9 hours. The reaction mixture is discharged onto 200 g of ice, adjusted to a pH of 6 using approximately 65 g of calcium carbonate, at a temperature of 0-5° C., and filtered. The filtrate contains the compounds of the formula (2-3b) in the form of the disodium salts, written—in the form of the free acids—thus:

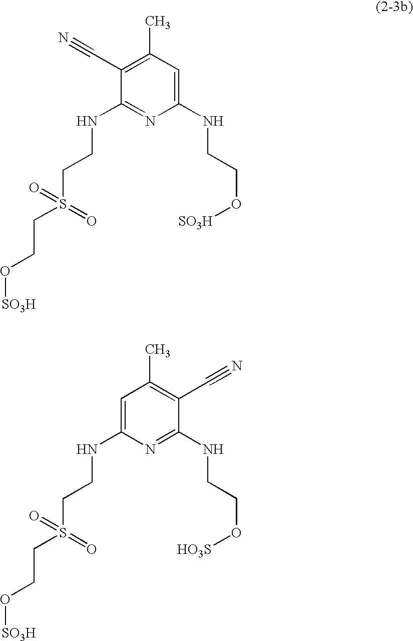

The resultant solution (the filtrate) is adjusted to a pH of 10 to 11 using 20% strength sodium hydroxide solution, stirred at this pH for 30 minutes, adjusted to a pH of 6 to 6.5 using hydrochloric acid, and concentrated by evaporation. This gives a product containing the compounds of the formula (2-3c) in the form of the sodium salts, written—in the form of the free acids—thus:

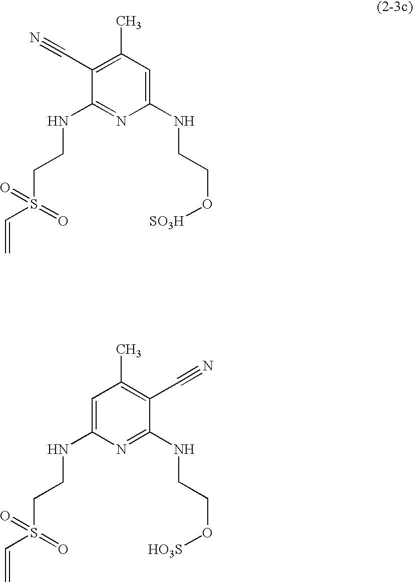

93.5 g of the reaction mixture of compounds of the formula (2-3a) are added to a reaction mixture of the diazonium salt obtained by conventionally diazotizing 26.9 g of 2-amino-5-(4-sulfophenylazo)benzensulfonic acid (di-yellow acid, D44). Using sodium carbonate solution, the pH of the mixture is held at 2 to 2.5 for approximately 4 hours. The reaction mixture is concentrated under reduced pressure, taken up in 400 ml of water, filtered and poured into 3 l of ethanol. The precipitated dye is filtered off with suction and dried. The resultant product contains the dyes of the formula (1-3a) in the form of the disodium salts, written—in the form of the free acids—thus:

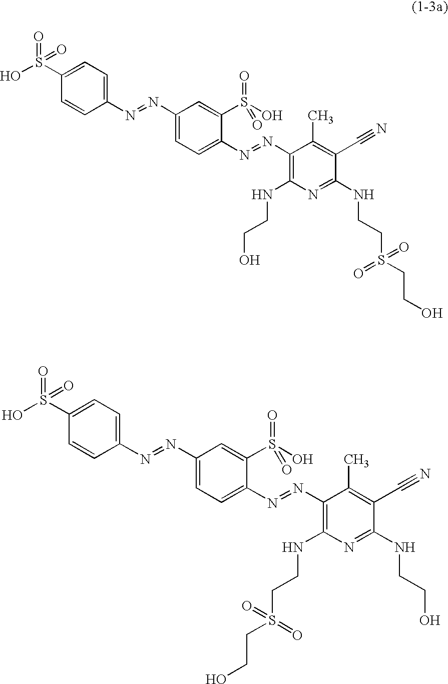

The resultant product is introduced at a temperature of 10-15° C. into 60 ml of 100% sulfuric acid (monohydrate) and the mixture is stirred at this temperature for 9 hours. The reaction mixture is discharged onto 400 g of ice, adjusted to a pH of 6 using 115 g of calcium carbonate, at a temperature of 0-5° C., and filtered. The filtrate contains the dyes of the formula (1-3b) in the form of the tetrasodium salts, written—in the form of the free acids—thus:

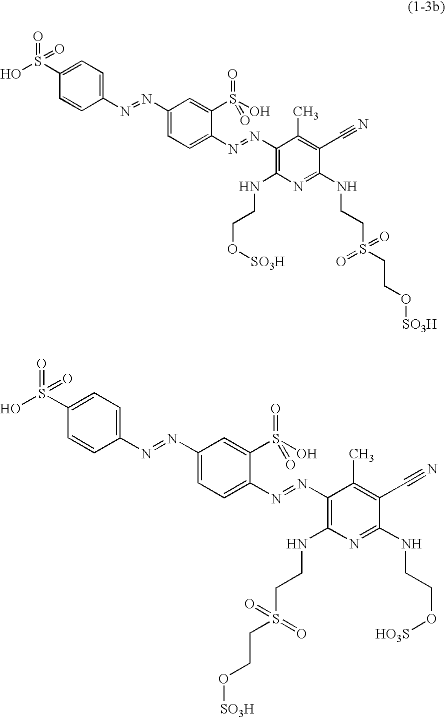

which dye polyamide in red shades. The dyeings are notable for good all-round fastnesses, particularly light and wet fastnesses.

Dyes of the formula (1-3b) can also be obtained by coupling the diazonium salt of di-yellow acid onto couplers of the formula (2-3b).

The solution of the dyes (the filtrate) is adjusted to a pH of 10 to 11 using 20% strength sodium hydroxide solution, stirred at this pH for 30 minutes, adjusted to a pH of 6 to 6.5 using hydrochloric acid, and concentrated by evaporation. This gives a product which contains the dyes of the formula (1-3c) in the form of the trisodium salts, written in the form of the free acids thus:

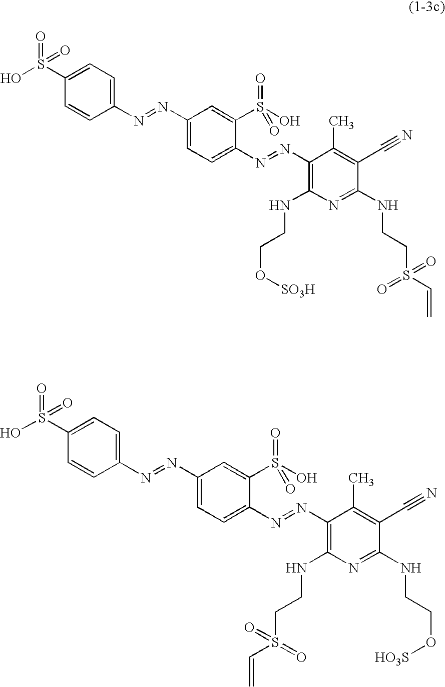

which dye polyamide in red shades. The dyeings are notable for good all-round fastnesses, particularly light and wet fastnesses.

Dyes of the formula (1-3c) can also be obtained by coupling the diazonium salt of di-yellow acid onto couplers of the formula (2-3c).

### EXAMPLE 4

81 g of the reaction mixture of example 3 formula (2-3a) are added to a reaction mixture of the diazonium salt obtained by conventionally diazotizing 25 g of 2-(4-amino-3-sulfophenyl)-2H-naphtho[1,2-d][1,2,3]triazole-6,8-disulfonic acid (formula D48).

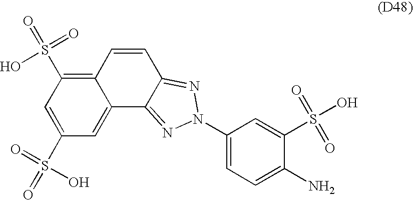

Using sodium carbonate solution, the pH of the mixture is held at 2 to 2.5 for approximately one hour. The reaction mixture is filtered and poured into 1.5 l of ethanol. The precipitated dye is filtered off with suction and dried. The resultant product contains the dyes of the formula (1-4a) in the form of the trisodium salts, written—in the form of the free acids—thus:

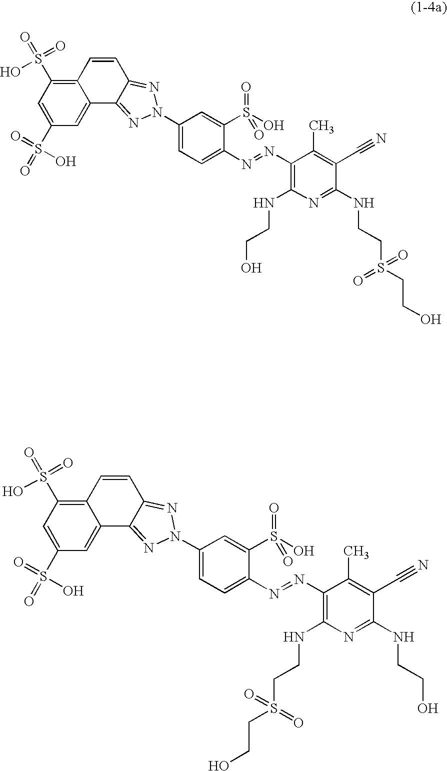

20 g of the resultant product are introduced at a temperature of 10-15° C. into 60 ml of 100% sulfuric acid (monohydrate) and the mixture is stirred at this temperature for 6 hours. The reaction mixture is discharged onto 400 g of ice, adjusted to a pH of 5.5 using 110 g of calcium carbonate, at a temperature of 0-5° C., and filtered. The filtrate contains the dyes of the formula (1-4b) in the form of the pentasodium salts, written—in the form of the free acids—thus:

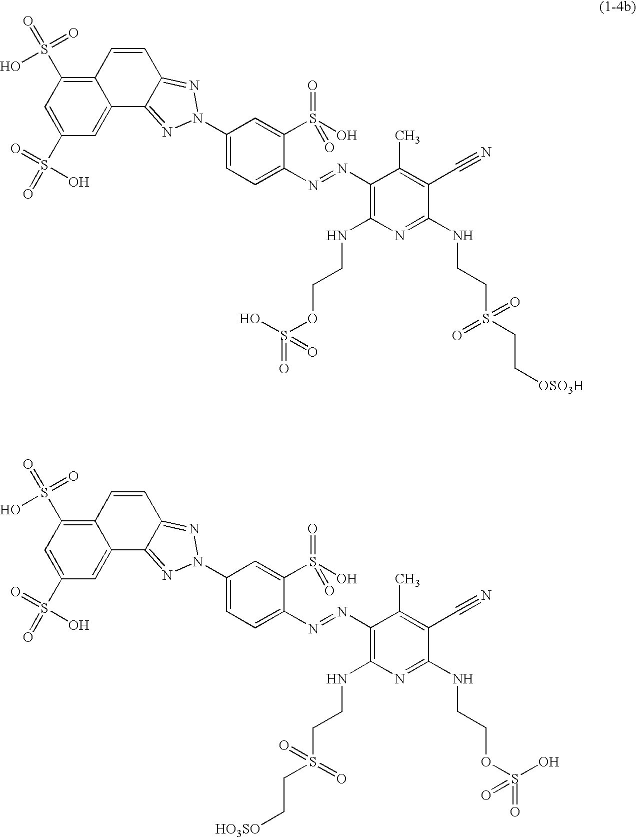

which dye polyamide in clear orange shades. The dyeings are notable for good all-round fastnesses, particularly light and wet fastnesses.

The solution of the dyes (the filtrate) is adjusted to a pH of 10 to 11 using 20% strength sodium hydroxide solution, stirred at this pH for 30 minutes, adjusted to a pH of 6 to 6.5 using hydrochloric acid, and concentrated by evaporation. This gives a product containing the dyes of the formula (1-4c) in the form of the tetrasodium salts, written—in the form of the free acids—thus:

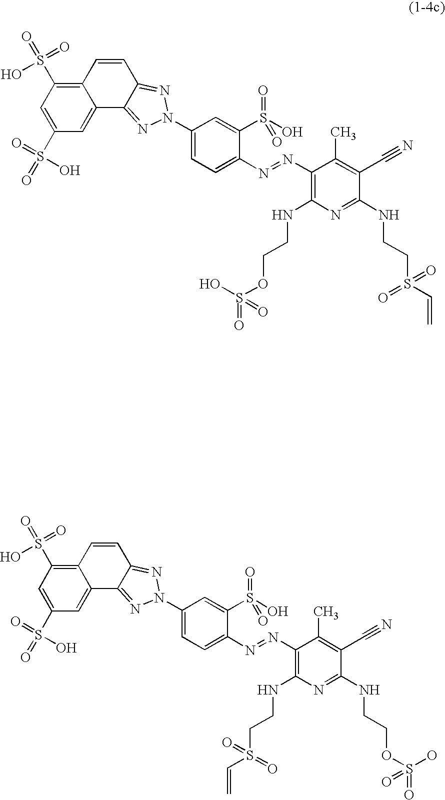

which dye polyamide in clear orange shades. The dyeings are notable for good all-round fastnesses, particularly light and wet fastnesses.

The compounds in the table below can be prepared in the same way as for the preceding examples:

TABLE 1

| | | | Dyeing | |
|---|---|---|---|---|
| | | | on | Dyeing |
| | | | poly- | on |
| Example | Formulae | | amide | cotton |
| 5 | 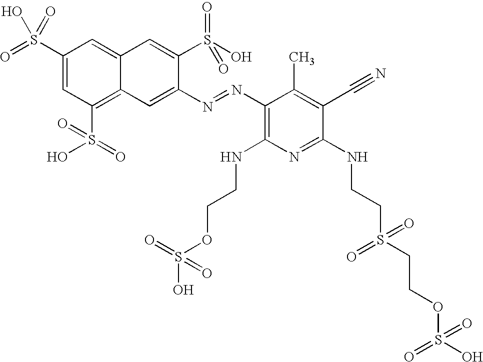 | 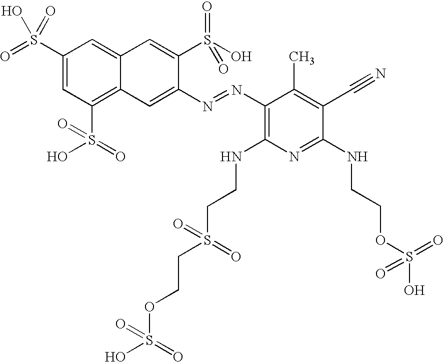 | goldenyellow | goldenyellow |
| 6 | 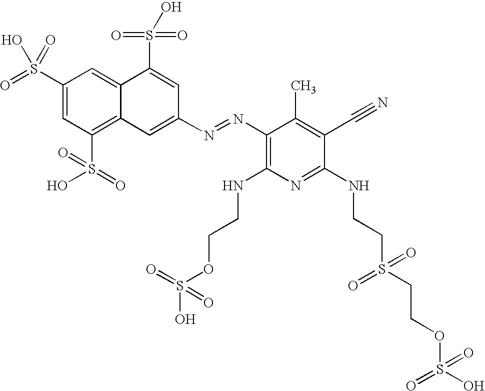 | 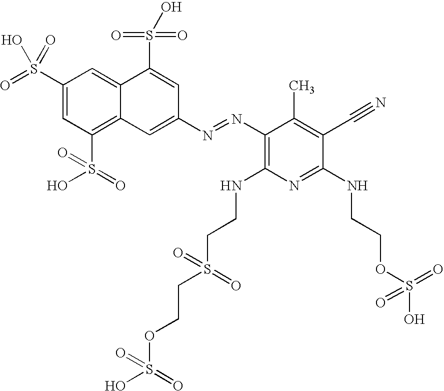 | yellow | yellow |
| 7 | 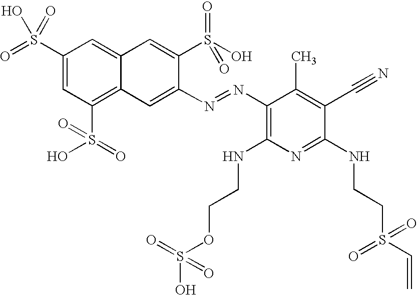 | 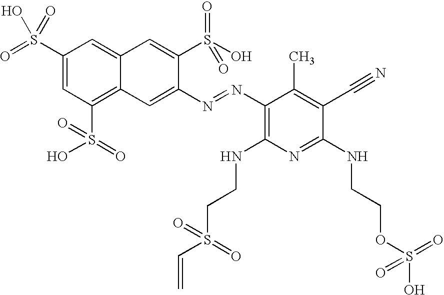 | yellow | yellow |
| 8 | 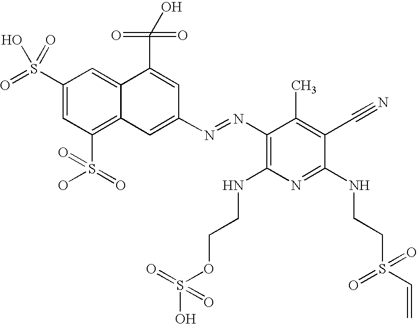 | 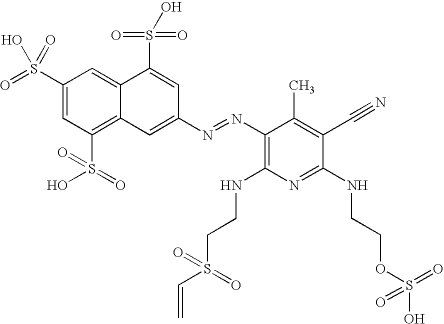 | yellow | yellow |
| 9 | 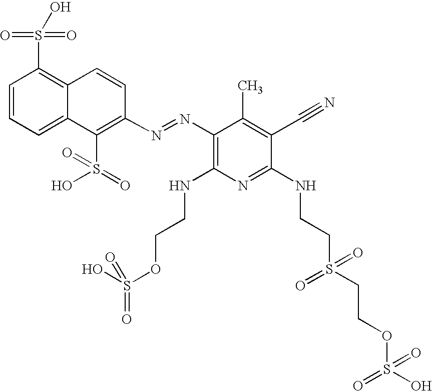 | 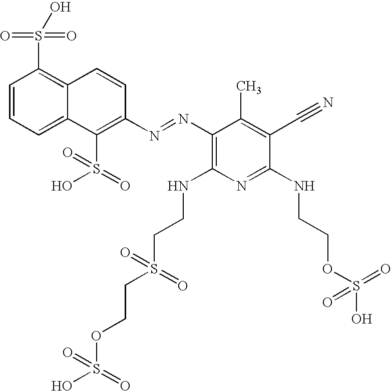 | yellow | yellow |
| 10 | 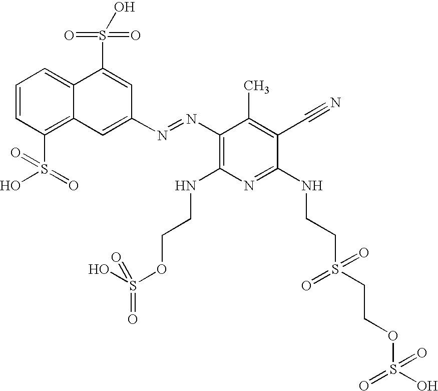 | 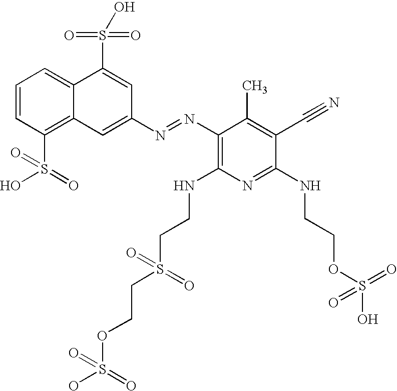 | yellow | yellow |
| 11 | 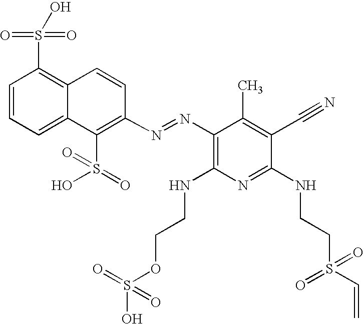 | 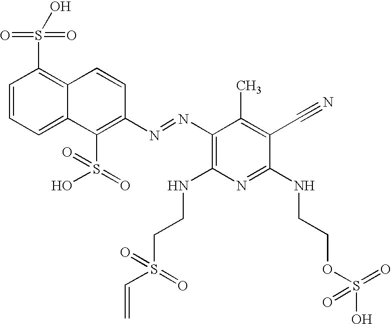 | yellow | yellow |
| 12 | 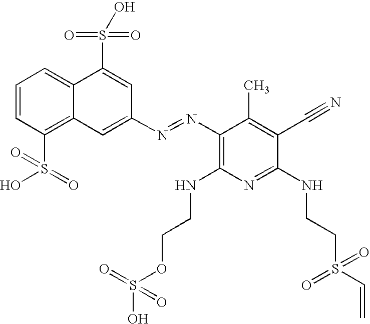 | 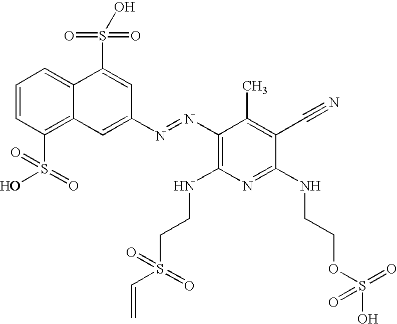 | yellow | yellow |
| 13 | 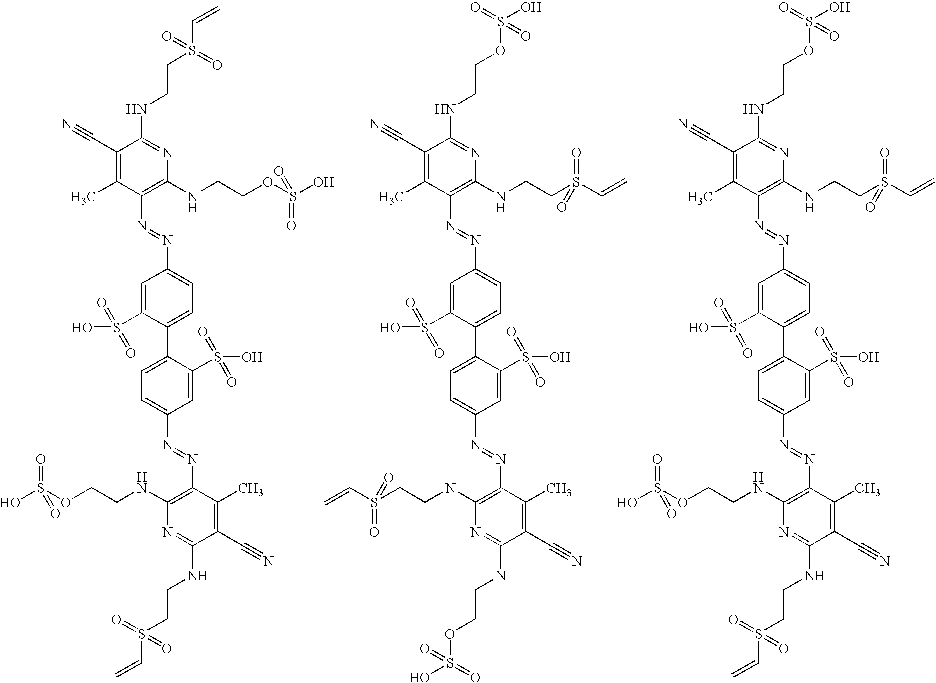 | | yellow | yellow |
| 14 | 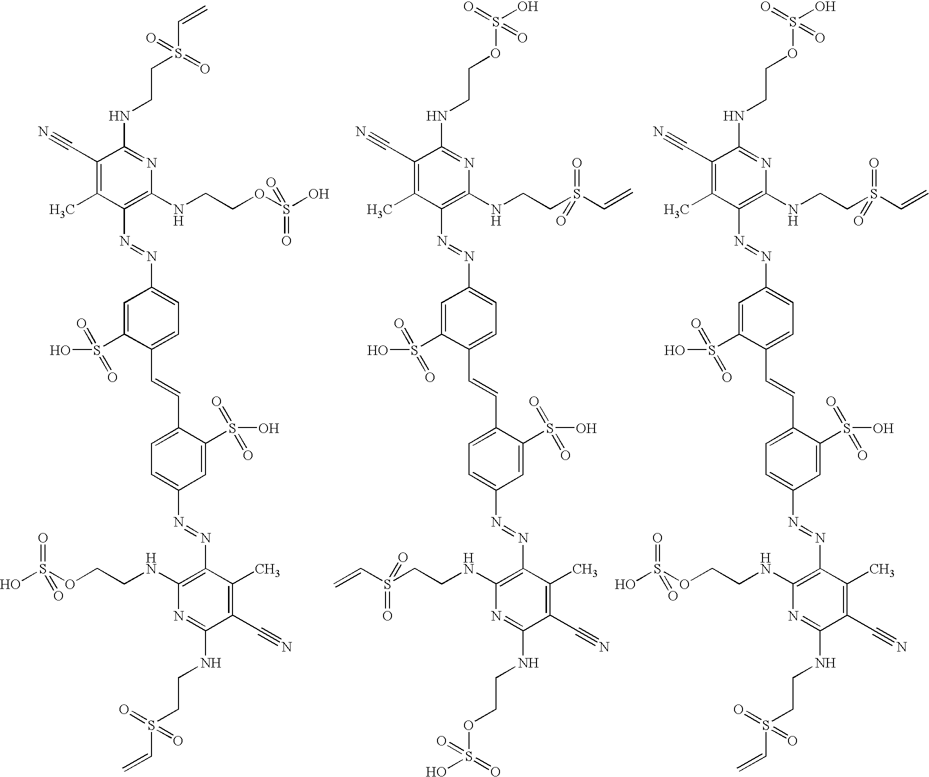 | | scarlet | scarlet |
| 15 | 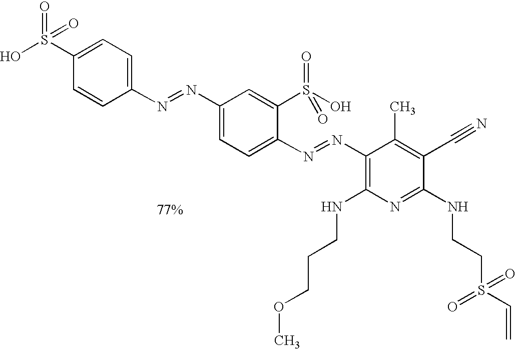 | 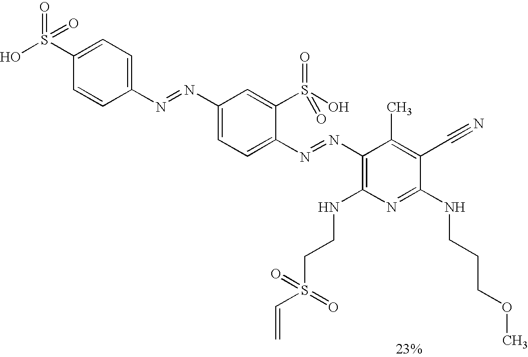 | red | red |
| 16 | 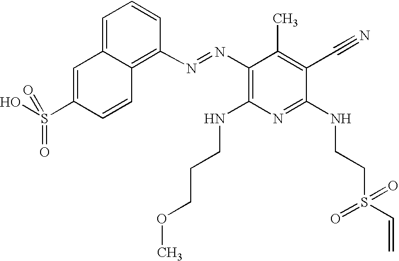 | 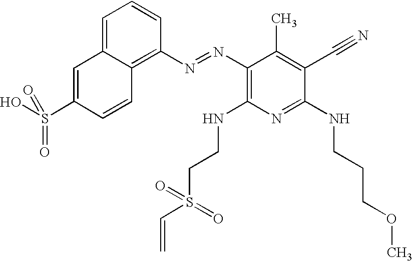 | amber | |
| 17 | 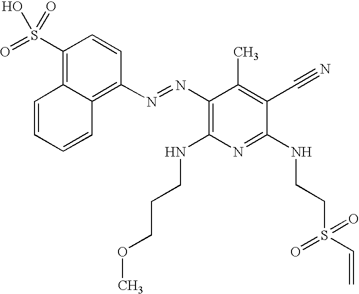 | 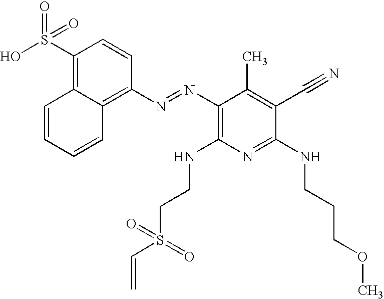 | orange | |
| 18 | 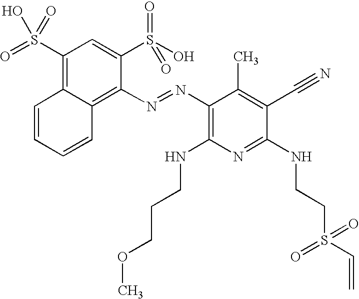 | 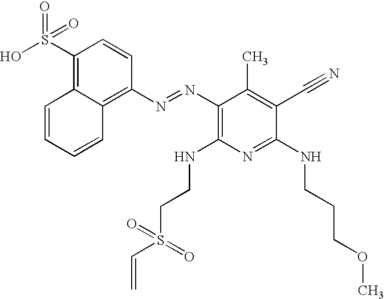 | orange | |
| 19 | 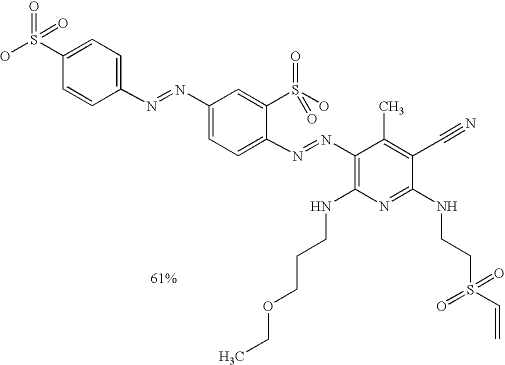 | 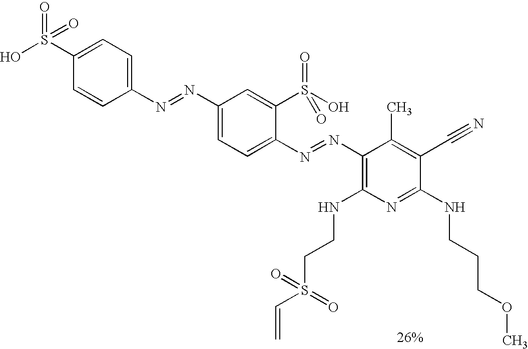 | red | red |
| 20 | 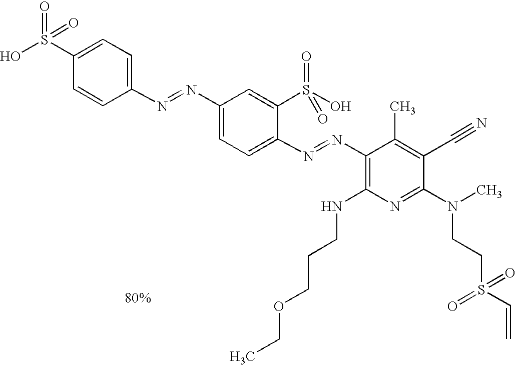 | 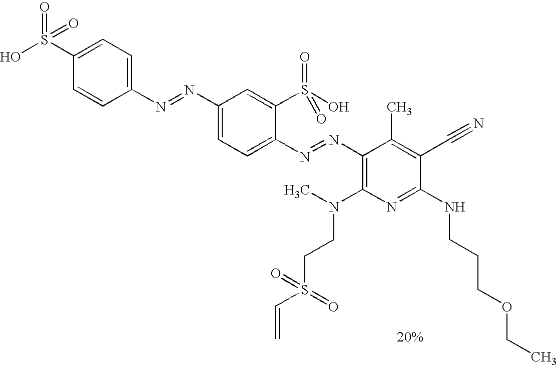 | red | red |
| 21 | 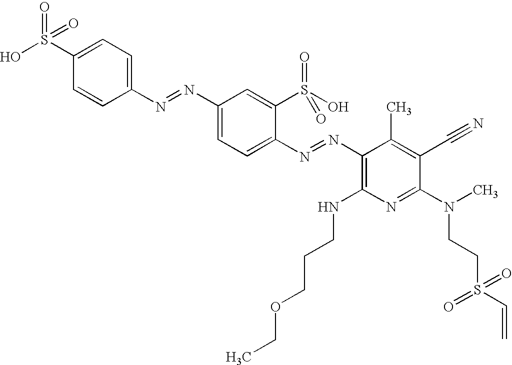 | 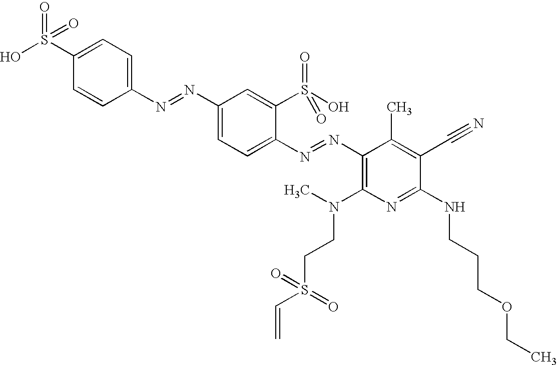 | red | red |
| 22 | 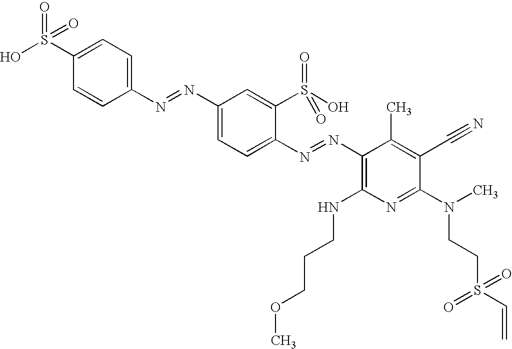 | 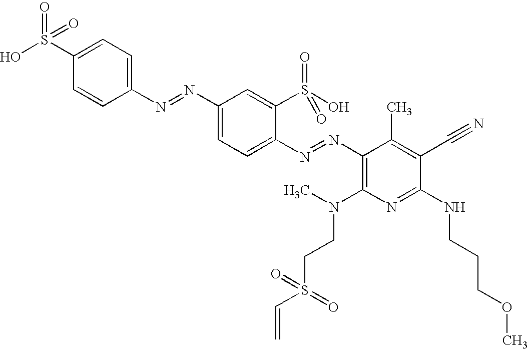 | red | red |
| 23 | 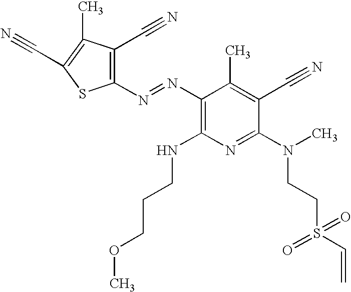 | 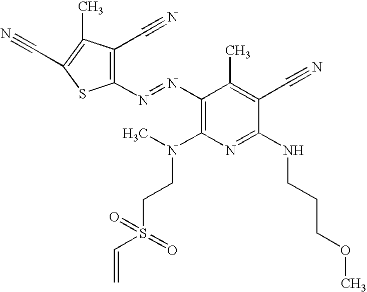 | red | red |
| 24 | 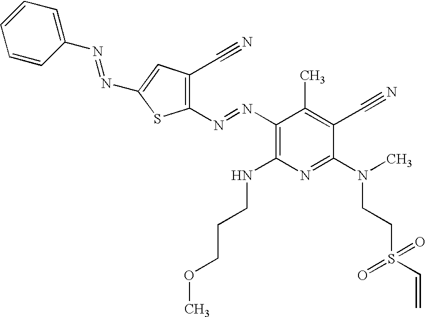 | 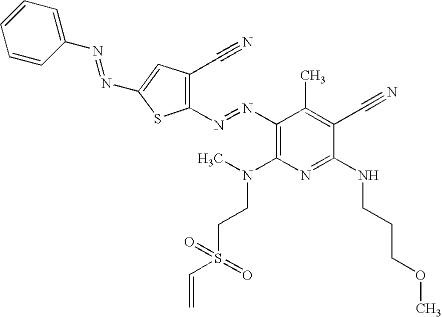 | blue | blue |
| 25 | 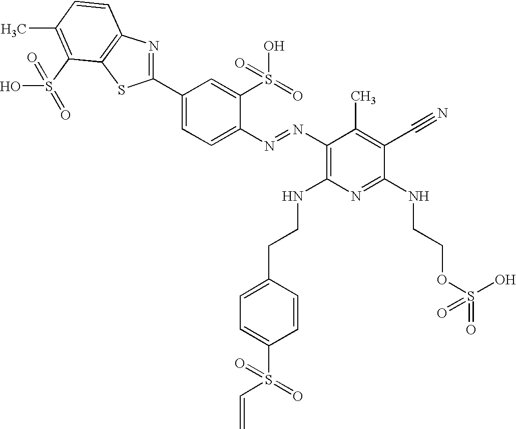 | 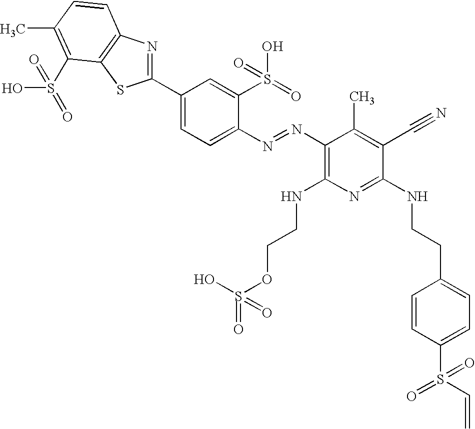 | orange | orange |
| 26 | 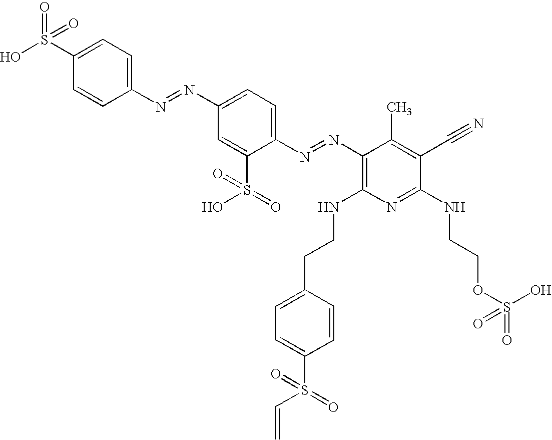 | 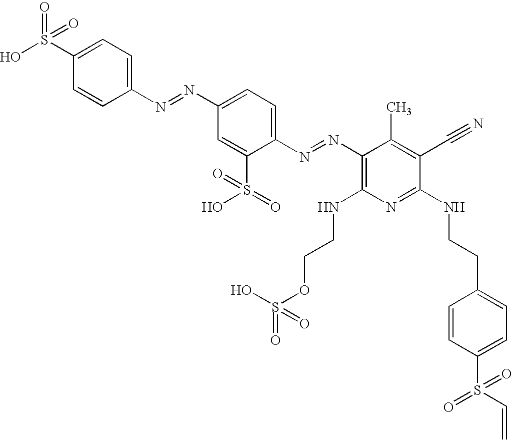 | red | red |
| 27 | 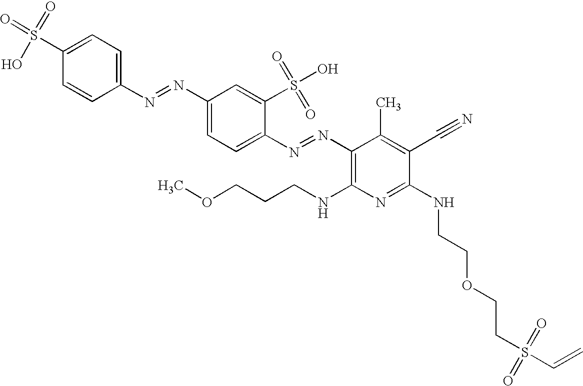 | 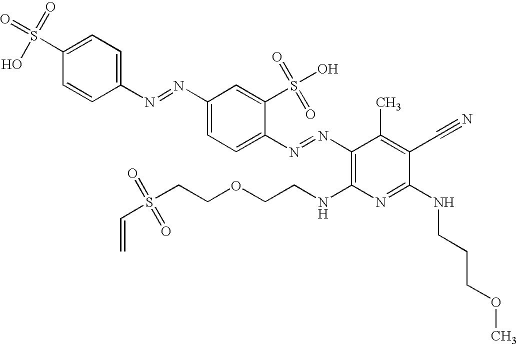 | red | red |
| 28 | 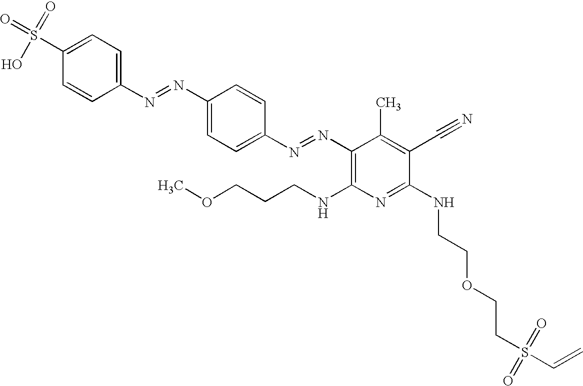 | 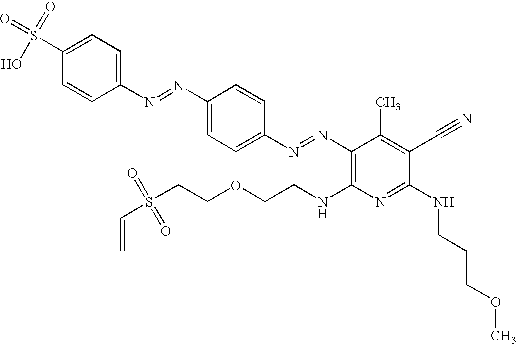 | red | red |
| 29 | 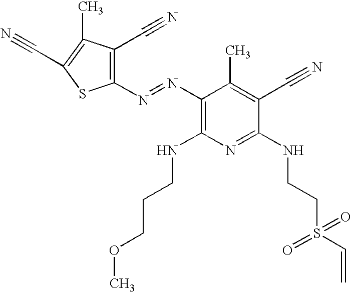 | 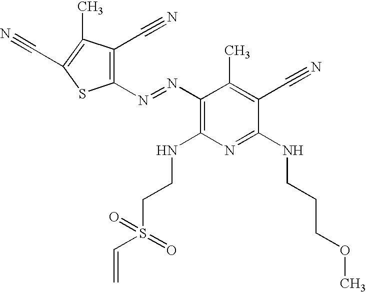 | red | red |
| 30 | 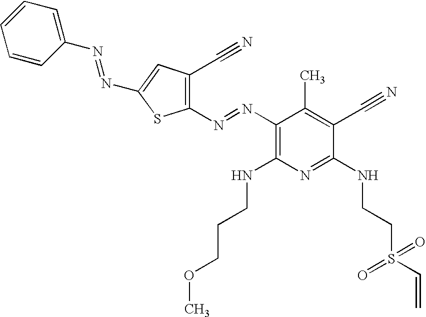 | 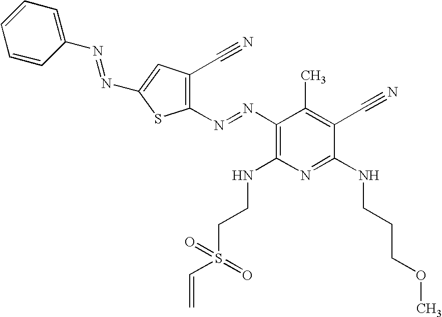 | blue | blue |
| 31 | 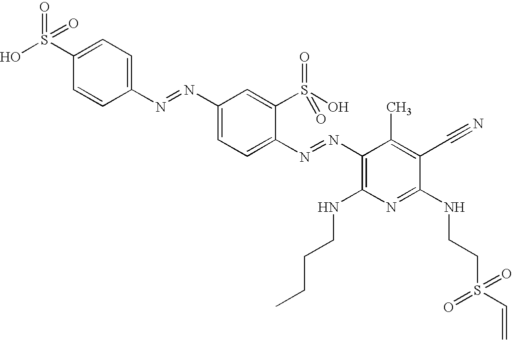 | 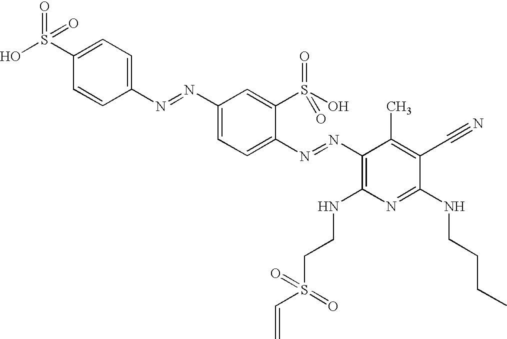 | red | red |
| 32 | 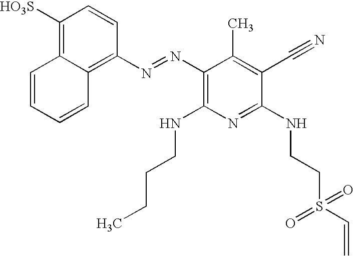 | 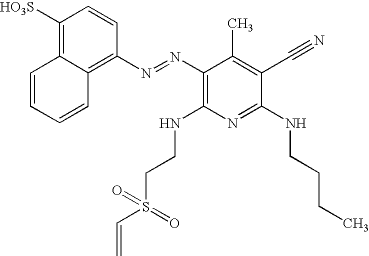 | orange | orange |
| 33 | 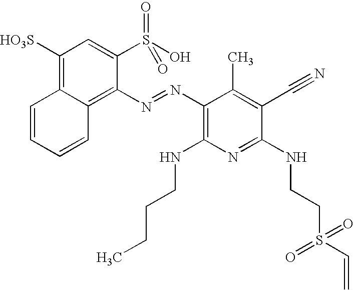 | 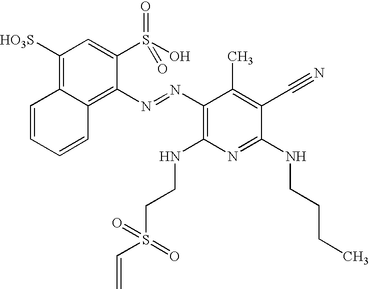 | orange | orange |
| 34 | 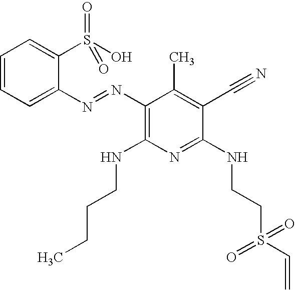 | 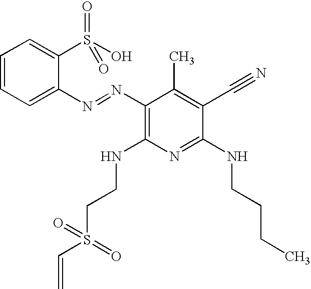 | yellow | yellow |
| 35 | 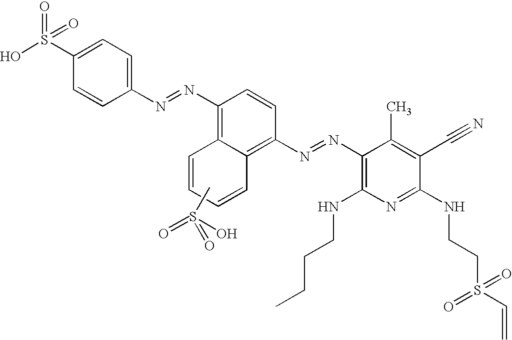 | 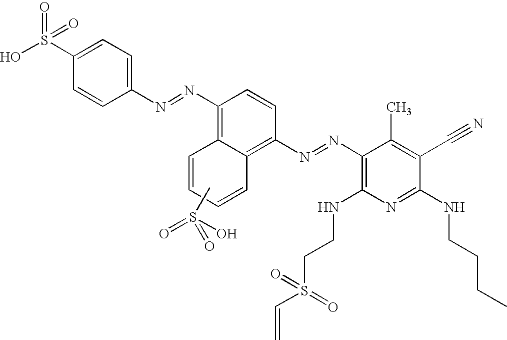 | violet | violet |
| 36 | 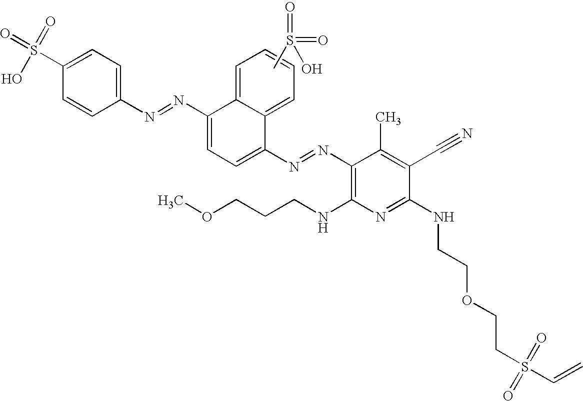 | 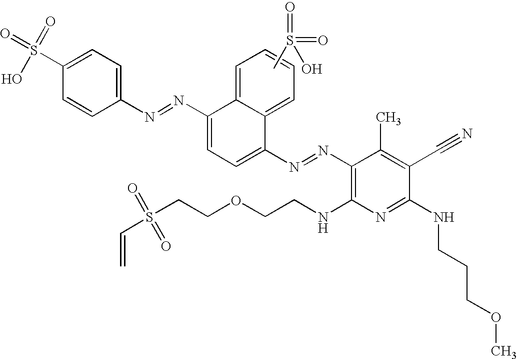 | violet | violet |
| 37 | 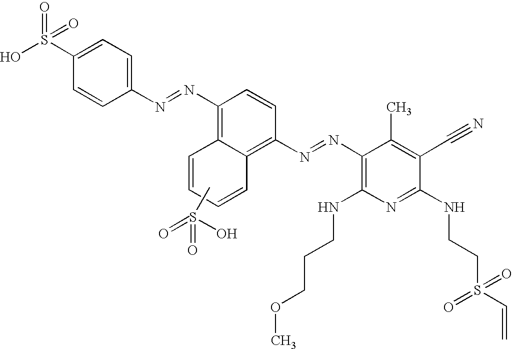 | 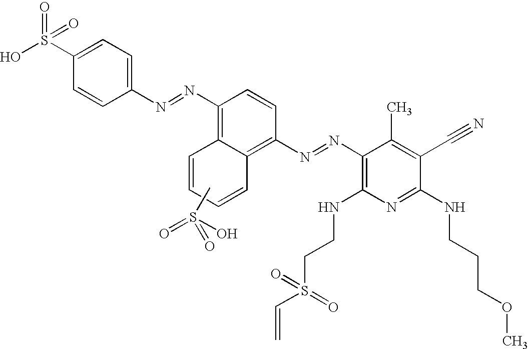 | violet | violet |
| 38 | 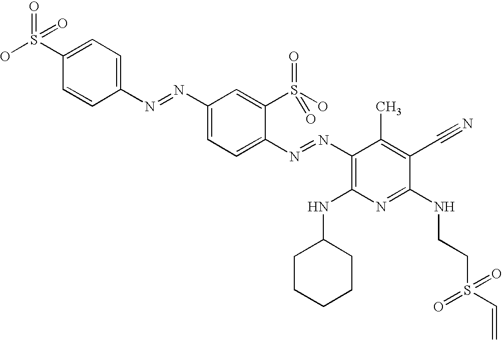 | 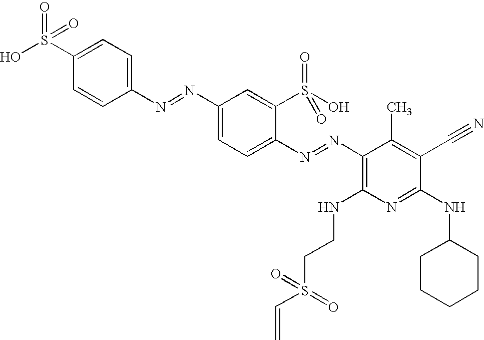 | red | red |
| 39 | 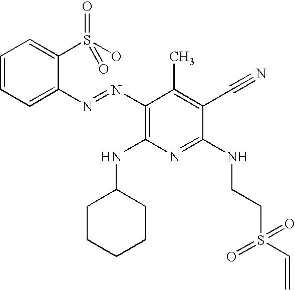 | 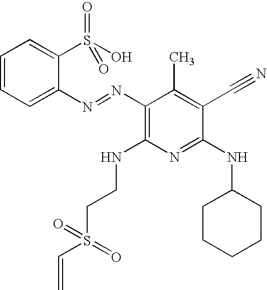 | yellow | yellow |
| 40 | 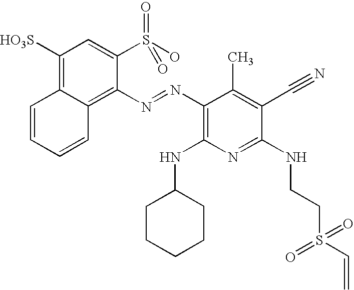 | 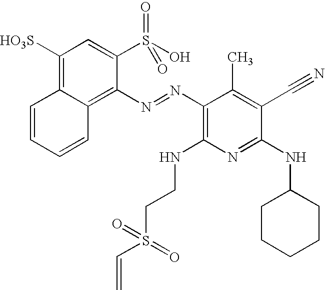 | orange | orange |
| 41 | 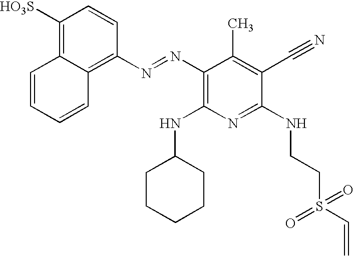 | 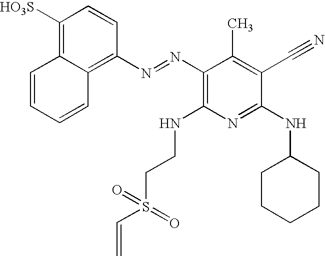 | orange | orange |

### EXAMPLE 42

Into 150 ml of 2-(2-aminoethylsulfanyl)ethanol there are introduced 47 g of 2,6-dichloro-4-methylnicotinonitrile (3-cyano-2,6-dichloro-4-methylpyridine) at a temperature of 50° C. The mixture is heated to 120° C. and stirred at this temperature for 6 hours. The reaction mixture is diluted with 500 ml of water, admixed with 70 g of sodium chloride and stirred for approximately 1 hour. When the mixture is allowed to stand an oil phase separates out and is conveyed onto ice. The mixture this produces is adjusted to a pH of 3 to 3.5 using hydrochloric acid, left to stand for approximately 16 hours and filtered with suction, and the presscake obtained is washed with water, suspended in water, filtered off again with suction and washed with water. The presscake obtained contains the product of the following formula (11-1).

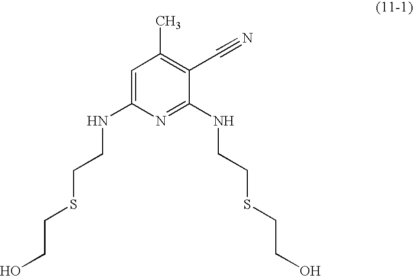

The presscake obtained is dispersed in 300 ml of ice-water. The mixture is heated to 30° C. and 1.65 g of sodium tungstate dihydrate are added. Added to the mixture at a temperature of 30° C. over the course of 30 minutes are 15 ml of 35% strength hydrogen peroxide solution, and over the course of 30 minutes 15 ml of a 35% strength hydrogen peroxide solution are added as well, the reaction mixture undergoing heating to 50° C. It is stirred at this temperature for 3 hours. Subsequently the oxidation is completed by dropwise addition of a total of 68 ml of 35% strength hydrogen peroxide solution with simultaneous raising of the temperature to 75° C. The reaction mixture (634 g) contains the product of the formula (12-1)

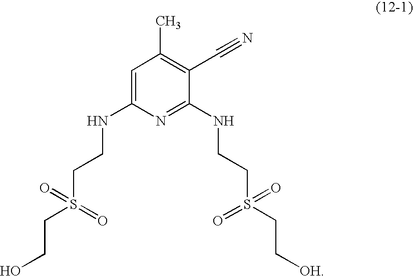

315 g of the resultant reaction mixture are added to a reaction mixture of the diazonium salt obtained by conventionally diazotizing 8.93 g of 2-amino-5-(4-sulfophenylazo)benzenesulfonic acid (di-yellow acid). Using sodium carbonate solution, the pH of the mixture is held at 3.5 to 4.5 for approximately 4 hours and then adjusted to 6.5. The reaction mixture is filtered and concentrated. The residue this produces contains the product of the formula (13-1a) in the form of the disodium salt, written—in the form of the free acid—thus:

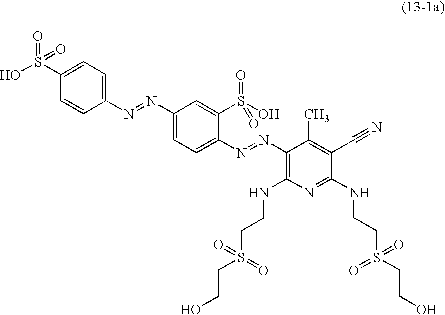

The resultant product is introduced at a temperature of 10-15° C. into 90 ml of 100% sulfuric acid (monohydrate) and the mixture is stirred at this temperature for 2 hours. The reaction mixture is discharged onto 600 g of ice, adjusted to a is pH of 6 using 170 g of calcium carbonate, at a temperature of 0-5° C., and filtered. Half of the filtrate is concentrated. The residue contains the product of the formula (13-1b) in the form of the tetrasodium salt, written—in the form of the free acid—thus:

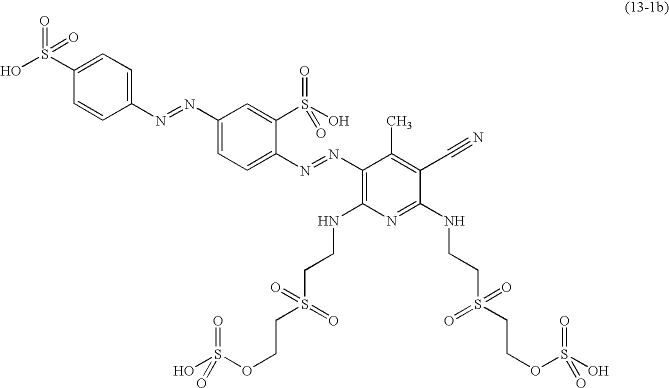

which dyes polyamide in red shades. The dyeings are notable for good all-round fastnesses, particularly light and wet fastnesses.

The remaining half of the filtrate is adjusted to a pH of 10 to 11 using 20% strength sodium hydroxide solution, stirred at this pH for one hour, adjusted to a to pH of 6 to 6.5 using hydrochloric acid, the product is isolated conventionally by salting out with sodium chloride, and the presscake obtained is dried. This gives the product of the formula (13-1c) in the form of the disodium salt, written—in the form of the free acid—thus:

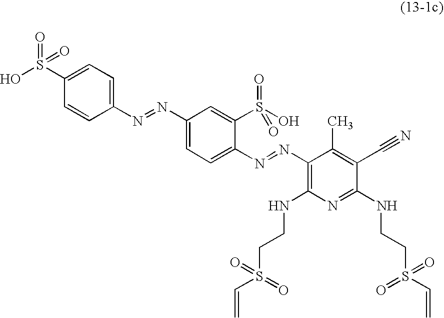

which dyes polyamide in red shades. The dyeings are notable for good all-round fastnesses, particularly light and wet fastnesses.

The compounds in the table below can be prepared in the same way as for the preceding example:

TABLE 2

| Example | Formulae |
|---|---|
| 43 | 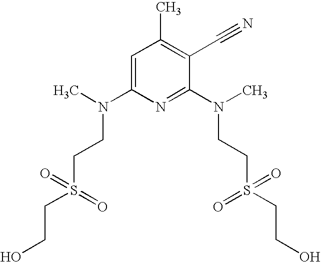 |
| 44 | 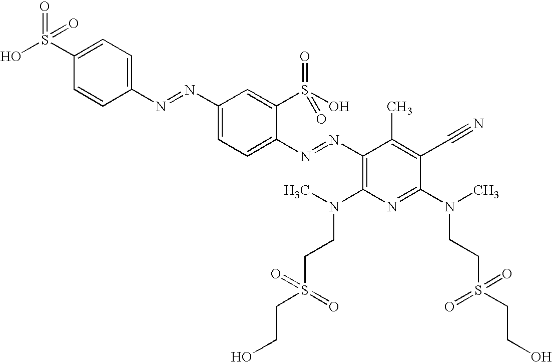 |
| 45 | 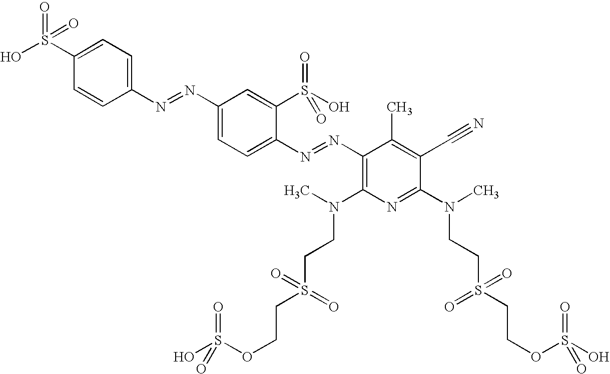 |
| 46 | 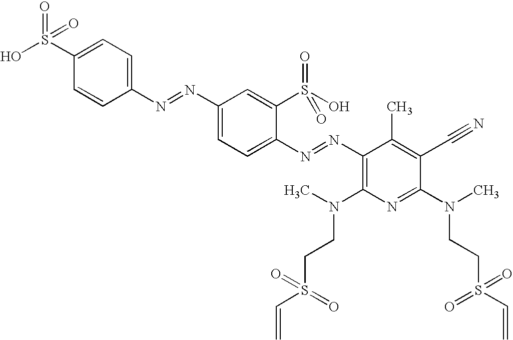 |

Further examples of dyes of the invention are set out in the following table:

TABLE 3

| Example | Formulae |
|---|---|
| 47 | 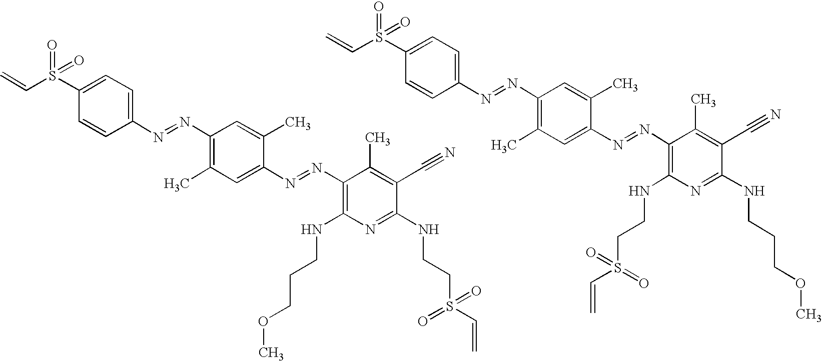 |
| 48 | 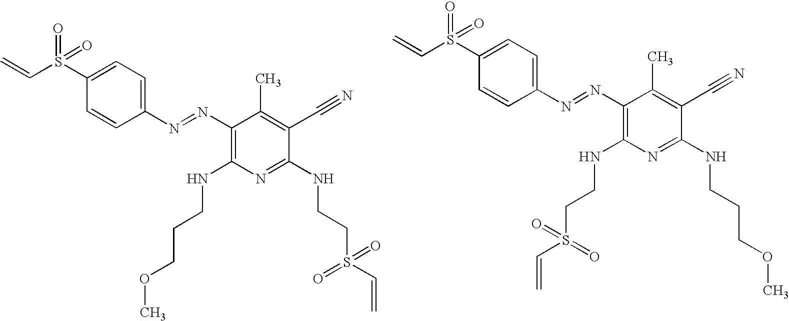 |
| 49 | 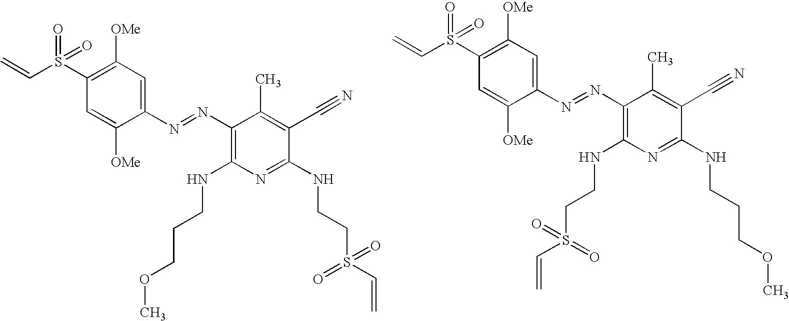 |
| 50 | 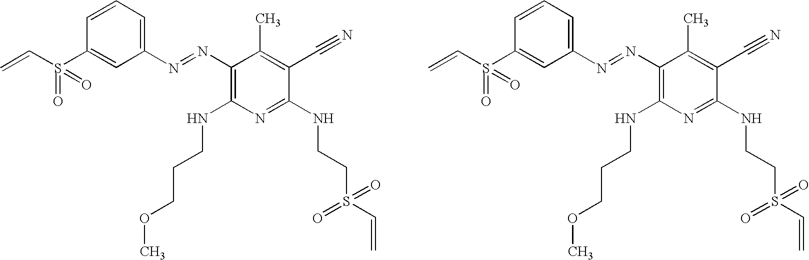 |
| 51 | 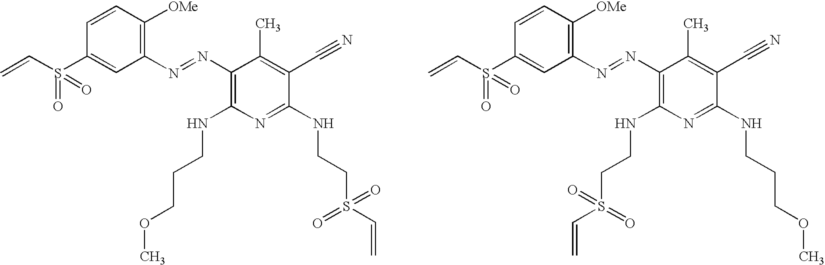 |
| 52 | 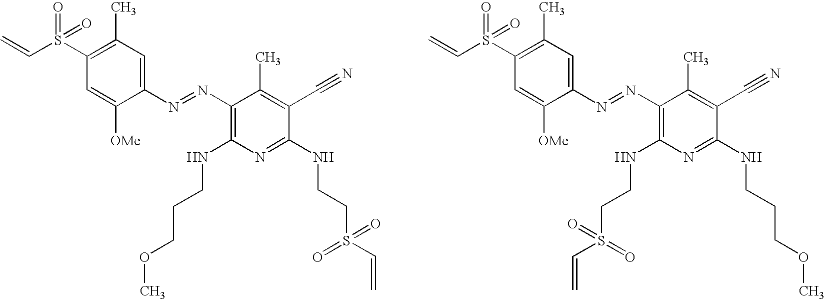 |
| 53 | 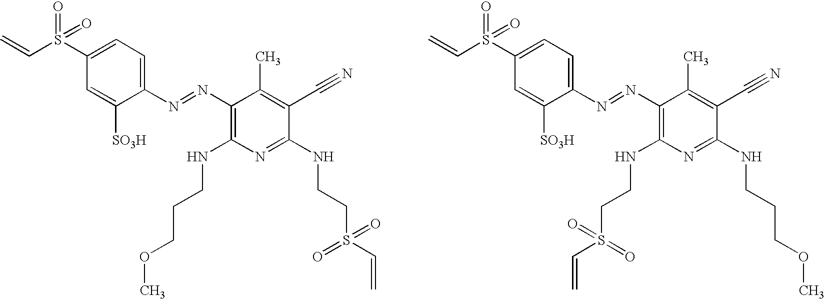 |
| 54 | 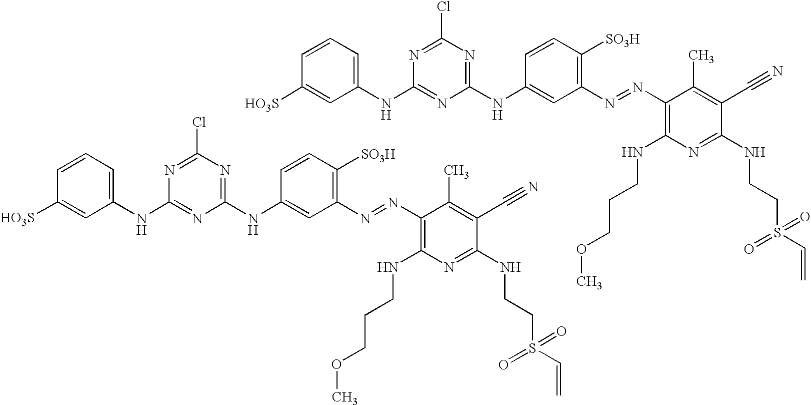 |
| 55 | 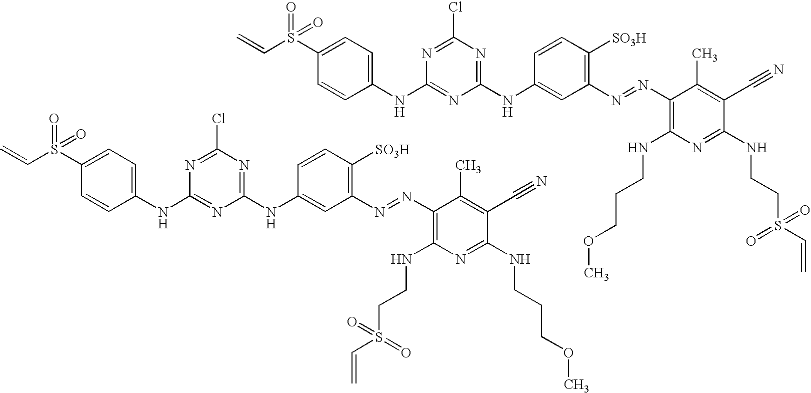 |
| 56 | 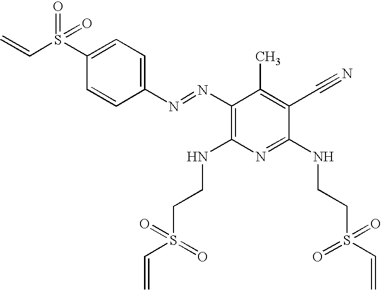 |
| 57 | 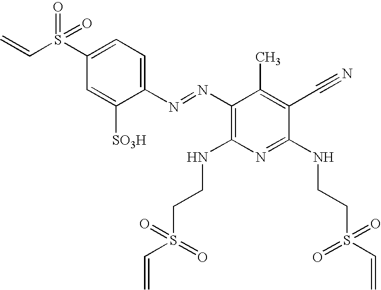 |
| 58 | 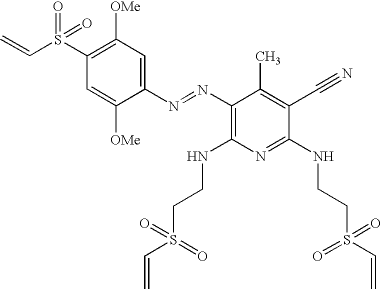 |
| 59 | 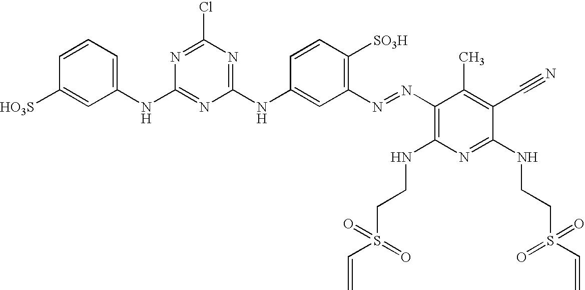 |
| 60 | 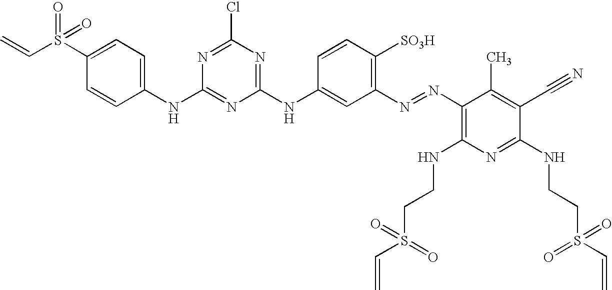 |

### Dyeing Example 1

1 part of the dye of the formula (1-1c) is dissolved in 2000 parts of water and 5 parts of sodium sulfate, and 1 part of a leveling assistant (based on a condensation product of a higher aliphatic amine and ethylene oxide) and 5 parts of sodium acetate are added.

The pH is then adjusted to 4.5 using acetic acid (80%). The dyebath is heated to 50° C. for 10 minutes and then entered with 100 parts of a woven wool fabric. The temperature is raised to 100° C. over the course of 50 minutes and then dyeing is carried out at this temperature for 60 minutes. This is followed by cooling to 90° C. and removal of the dyed material. The wool fabric is washed with hot and cold water, then spun and dried, The red dyeing obtained has good light and wet fastnesses and also good levelness in the fiber.

### Dyeing Example 2

1 part of the dye of the formula (1-2c) is dissolved in 2000 parts of water and 1 part of a leveling assistant (based on a condensation product of a higher aliphatic amine and ethylene oxide) and 6 parts of sodium acetate are added. The pH is then adjusted to 5 using acetic acid (80%). The dyebath is heated to 50° C. for 10 minutes and then entered with 100 parts of a woven polyamide fabric. The temperature is raised to 110° C. over the course of 50 minutes and then dyeing is carried out at this temperature for 60 minutes. This is followed by cooling to 60° C. and removal of the dyed material. The polyamide fabric is washed with hot and cold water, soaped and then spun and dried. The red dyeing obtained has good light and wet fastnesses and also good levelness in the fiber,

### Dyeing Example 3

2 parts of a dye obtained as per formula (1-3b) and 50 parts of sodium chloride are dissolved in 999 parts of water, and 5 parts of sodium carbonate, 0.7 part 30 of sodium hydroxide (in the form of a 32.5% strength aqueous solution) and optionally 1 part of a wetting agent are added. This dyebath is entered with 100 g of a woven cotton fabric. The temperature of the dyebath is first held at 25° C. for 10 minutes and then raised to the final temperature (40-80° C.) over 30 minutes, this temperature being maintained for a further 60-90 minutes. Thereafter the dyed goods are initially rinsed with tapwater for 2 minutes and then with deionized water for 5 minutes. The dyed goods are neutralized at 40° C. in 1000 parts of an aqueous solution containing 1 part of 50% strength acetic acid, for 10 minutes. The goods are rinsed again with deionized water at 70° C. and then soaped off at the boil with a laundry detergent for 15 minutes, rinsed once more and dried. The strong red dyeing obtained has very good fastness properties.

### Dyeing Example 4

A textile fabric consisting of mercerized cotton is padded with a liquor containing 35 g/l of calcium sodium carbonate, 100 g/l of urea and 150 g/l of a low-viscosity Na alginate solution (6%), and then dried. The liquor pickup is 70%.

The textile thus pretreated is printed with an aqueous ink containing

2% of the dye of example (1-3c)
20% of sulfolane

### 0.01% of Mergal K9N

77.99% of water
using a drop-on-demand (bubblejet) ink-jet printing head, The print is fully dried. It is fixed by means of saturated steam at 102° C. for 8 minutes. The print is then rinsed warm, subjected to a fastness wash with hot water at 95° C., rinsed warm and then dried. The result is a bluish red print having excellent wearing fastnesses.

1-22. (canceled)

23. A reactive dye of formula (1) 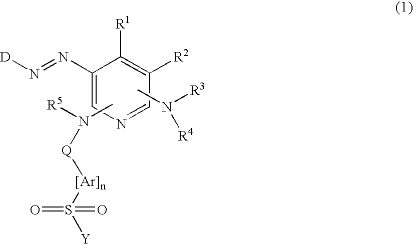
wherein
D is the radical of a diazo component of the benzene or naphthalene series, a heterocyclic radical, or the radical of a monoazo or diazo dye;
R¹ is C₁ to C₄ alkyl;
R² is cyano, carbamoyl, or sulfomethyl;
R³, R⁴, and R⁵
are, independently of one another, hydrogen, C₁ to C₁₈ alkyl, or C₄ to C₇ cycloalkyl, wherein said alkyl and cycloalkyl is optionally substituted by hydroxyl, sulfo, sulfato, or vinylsulfonyl and wherein, except for methyl, one or more nonadjacent carbon atoms is optionally replaced by oxygen or NR⁷, or R³ and R⁴ are part of heterocyclic groups;
R⁷ is hydrogen or C₁ to C₄ alkyl;
Q is C₁ to C₁₈ alkylene optionally substituted by C₁ to C₄ alkyl, halogen, or hydroxyl and wherein, except for methylene, one or more nonadjacent carbon atoms is optionally replaced by oxygen or NR⁷;
n is 0 or 1;
Ar is a phenylene or naphthylene radical optionally substituted by sulfo, carboxyl, C₁ to C₄ alkyl, C₁ to C₄ alkoxy, or halogen; and
Y is —CH═CH₂ or —CH₂CH₂Y¹, wherein Y¹ is hydroxyl or a group which can be eliminated under the action of alkali.

24. The reactive dye of claim 23, wherein
D is selected from the group consisting of formula (D-I), (D-II), and (D-XII): 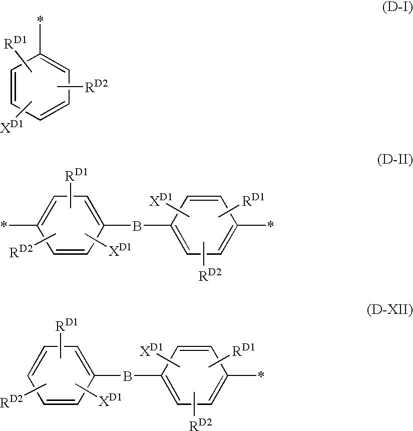
wherein
-* is the bond to the diazo group on the diaminopyridine group;
B is a direct chemical bond, an ethylene group, or a diazo group;
R^{D1} and R^{D2}
are, independently of one another, hydrogen, C₁ to C₄ alkyl, C₁ to C₄ alkoxy, hydroxyl, sulfo, carboxyl, cyano, nitro, amido, ureido, or halogen; and
X^{D1} is hydrogen, C₁ to C₄ alkyl, C₁ to C₄ alkoxy, hydroxyl, sulfo, carboxyl, cyano, nitro, amido, ureido, halogen, or a group of formula —SO₂-Z, wherein Z is —CH═CH₂ or —CH₂CH₂Y¹, wherein Y¹ is hydroxyl or a group which can be eliminated under the action of alkali; or
is a naphthyl group of formula (D-III): 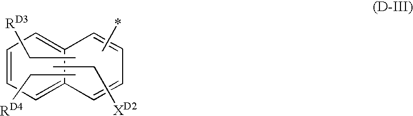
wherein
-* is the bond of the diazo group on the diaminopyridine group;
R^{D3} and R^{D4}
are, independently of one another, hydrogen, C₁ to C₄ alkyl, C₁ to C₄ alkoxy, hydroxyl, sulfo, carboxyl, cyano, nitro, amido, ureido, or halogen; and
X^{D2} is hydrogen, C₁ to C₄ alkyl, C₁ to C₄ alkoxy, hydroxyl, sulfo, carboxyl, cyano, nitro, amido, ureido, halogen, or a group of formula —SO₂-Z, wherein Z is —CH═CH₂ or —CH₂CH₂Y′, wherein Y¹ is hydroxyl or a group which can be eliminated under the action of alkali; or
is a group of formula (D-IV): 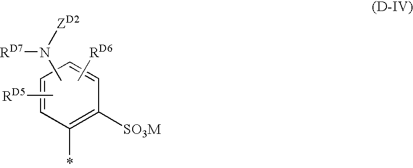
wherein
-* is the bond to the diazo group on the diaminopyridine group;
M is hydrogen, an alkali metal, ammonium, one equivalent of an alkaline earth metal, or a monovalent organic cation;
R^{D5} and R^{D6}
are, independently of one another, hydrogen, C₁ to C₄ alkyl, C₁ to C₄ alkoxy, hydroxyl, sulfo, carboxyl, cyano, nitro, amido, ureido, or halogen;
R^{D7} is hydrogen, C₁ to C₄ alkyl, or phenyl, wherein said phenyl is optionally substituted by C₁ to C₄ alkyl, C₁ to C₄ alkoxy, sulfo, halogen, or carboxyl; and
Z^{D2} is a group of formula (D-V), (D-VI) or (D-VII): 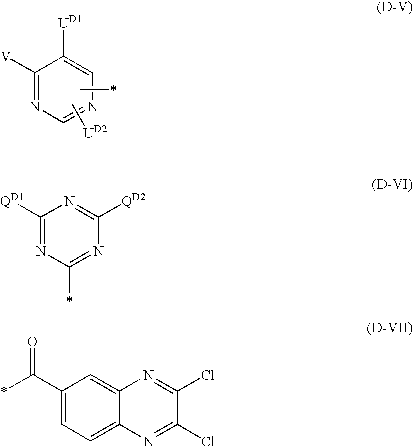
wherein
-* is the bond to nitrogen;
V is fluorine or chlorine;
U^{D1} and U^{D2}
are, independently of one another, fluorine, chlorine, or hydrogen; and
Q^{D1} and Q^{D2}
are, independently of one another, chlorine, fluorine, cyanamide, hydroxyl, C₁ to C₆ alkoxy, phenoxy, sulfophenoxy, mercapto, C₁ to C₆ alkylmercapto, pyridino, carboxypyridino, carbamoylpyridino, or a group of formula (D-VIII) or (D-IX): 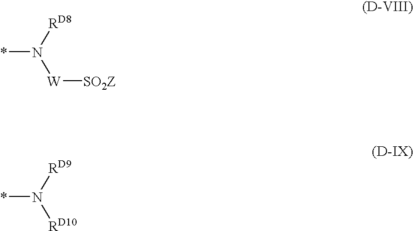
wherein
-* is the bond to the heterocycle;
R^{D8} is hydrogen, C₁ to C₆ alkyl, sulfo-C₁ to C₆ alkyl, or phenyl, wherein said phenyl is optionally substituted by C₁ to C₄ alkyl, C₁ to C₄ alkoxy, sulfo, halogen, carboxyl, acetamido, or ureido;
R^{D9} and R^{D10}
are, independently of one another, hydrogen, C₁ to C₆ alkyl, sulfo-C₁ to C₆ alkyl, or phenyl, wherein said phenyl is optionally substituted by C₁ to C₄ alkyl, C₁ to C₄ alkoxy, sulfo, halogen, carboxyl, acetamido, ureido, or define a cyclic ring system of formula —(CH₂)ⱼ—, wherein j is 4 or 5, or —(CH₂)₂-E-(CH₂)₂—, wherein E is oxygen, sulfur, sulfo, or —NR^{D11}, wherein R^{D11} is C₁ to C₆ alkyl;
W is phenylene optionally substituted by 1 or 2 substituents; C₁ to C₄ alkylenearylene or C₂ to C₆ alkylene, wherein one or more nonadjacent carbon atoms of said alkylenearylene or alkylene is optionally replaced by oxygen, sulfur, sulfo, amino, carbonyl, or carboxamide; phenylene-CONH-phenylene optionally substituted by C₁ to C₄ alkyl, C₁ to C₄ alkoxy, hydroxyl, sulfo, carboxyl, amido, ureido, or halogen; or naphthylene optionally substituted by one or two sulfo groups; and
Z wherein Z is —CH═CH₂ or —CH₂CH₂Y¹, wherein Y¹ is hydroxyl or a group which can be eliminated under the action of alkali; or
is a group of formula (D-X) or (D-XI): 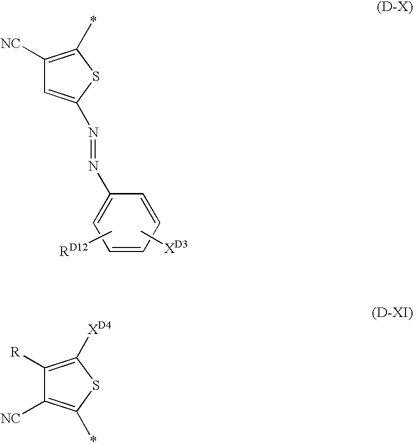
wherein
-* is the bond to the diazo group on the diaminopyridine group;
R^{D12} is hydrogen, C₁ to C₄ alkyl, C₁ to C₄ alkoxy, hydroxyl, sulfo, carboxyl, cyano, nitro, amido, ureido, or halogen;
X^{D3} is hydrogen, C₁ to C₄ alkyl, C₁ to C₄ alkoxy, hydroxyl, sulfo, carboxyl, cyano, nitro, amido, ureido, halogen, or a group of formula —SO₂-Z, wherein Z is —CH═CH₂ or —CH₂CH₂Y¹, wherein Y¹ is hydroxyl or a group which can be eliminated under the action of alkali;
X^{D4} is —CN or COOR^{D13}, wherein R^{D13} is C₁ to C₄ alkyl.

25. The reactive dye of claim 24, wherein said reactive dye has formula (1a): 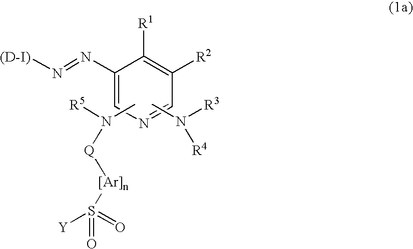
wherein n is 0 or 1.

26. The reactive dye of claim 24, wherein said reactive dye has formula (1 b): 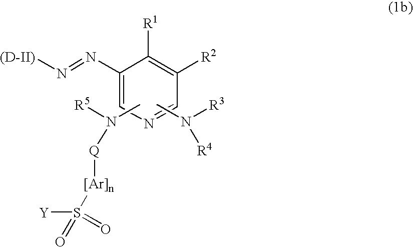
wherein n is 0 or 1.

27. The reactive dye of claim 24, wherein said reactive dye has formula (1c): 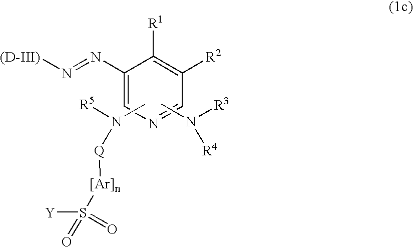
wherein n is 0 or 1.

28. The reactive dye of claim 24, wherein said reactive dye has formula (1d): 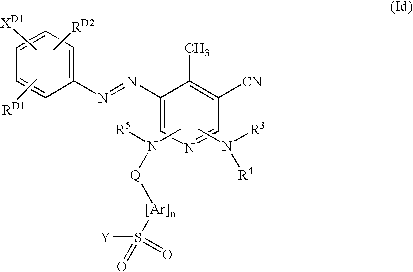
wherein
X^{D1} is hydrogen, C₁ to C₄ alkyl, C₁ to C₄ alkoxy, hydroxyl, sulfo, carboxyl, cyano, nitro, amido, ureido, or halogen; and
is hydrogen or C₁ to C₁₈ alkyl.

29. The reactive dye of claim 24, wherein said reactive dye has formula (1e): 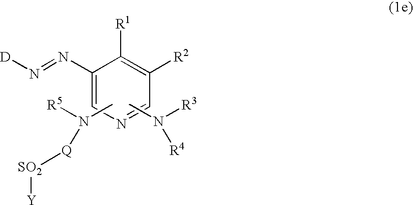
wherein
R¹ is C₁ to C₄ alkyl;
R² is cyano, carbamoyl, or sulfomethyl;
R³ and R⁴
are, independently of one another, hydrogen, C₁ to C₁₈ alkyl, or C₄ to C₇ cycloalkyl, wherein said alkyl or cycloalkyl is optionally substituted by hydroxyl, sulfo, sulfato, or vinylsulfonyl, and wherein, except for methyl, one or more nonadjacent carbon atoms is optionally replaced by oxygen or NR⁷, or R³ and R⁴ are part of heterocyclic groups;
R⁷ is hydrogen or C₁ to C₄ alkyl;
R⁵ is hydrogen or C₁ to C₁₈ alkyl;
Q is C₁ to C₁₈ alkylene optionally substituted by C₁ to C₄ alkyl, halogen, or hydroxyl, and wherein, except for methylene, one or more nonadjacent carbon atoms is optionally replaced by oxygen or NR⁷.

30. The reactive dye of claim 24, wherein said reactive dye has formula (1f): 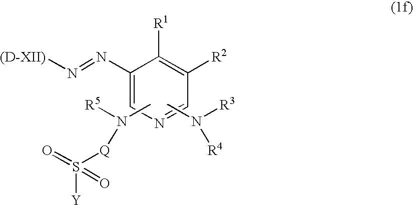
wherein
R¹ is C₁ to C₄ alkyl;
R² is cyano, carbamoyl, or sulfomethyl;
R³ and R⁴
are, independently of one another, hydrogen, C₁ to C₁₈ alkyl, or C₄ to C₇ cycloalkyl, wherein said alkyl or cycloalkyl is optionally substituted by hydroxyl, sulfo, sulfato, or vinylsulfonyl, and wherein, except for methyl, one or more nonadjacent carbon atoms is optionally replaced by oxygen or NR⁷, or R³ and R⁴ are part of heterocyclic groups;
R⁷ is hydrogen or C₁ to C₄ alkyl;
R⁵ is hydrogen or C₁ to C₁₈ alkyl;
Q is C₁ to C₁₈ alkylene optionally substituted by C₁ to C₄ alkyl, halogen, or hydroxyl, and wherein, except for methylene, one or more nonadjacent carbon atoms is optionally replaced by oxygen or NR⁷.

31. A diaminopyridine of formula (2): 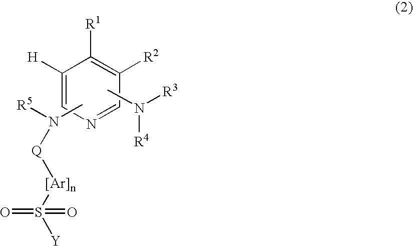
wherein
R¹ is C₁ to C₄ alkyl;
R² is cyano, carbamoyl, or sulfomethyl;
R³, R⁴, and R⁵
are, independently of one another, hydrogen, C₁ to C₁₈ alkyl, or C₄ to Cal cycloalkyl, wherein said alkyl or cycloalkyl is optionally substituted by hydroxyl, sulfo, sulfato, or vinylsulfonyl, and wherein, except for methyl, one or more nonadjacent carbon atoms is optionally replaced by oxygen or NR⁷, or R³ and R⁴ are part of heterocyclic groups;
R⁷ is hydrogen or C₁ to C₄ alkyl;
Q is C₁ to C₁₈ alkylene optionally substituted by C₁ to C₄ to alkyl, halogen, or hydroxyl and wherein, except for methylene, one or more nonadjacent carbon atoms is optionally replaced by oxygen or NR⁷,
n is 0 or 1;
Ar is a phenylene or naphthylene radical optionally substituted by sulfo, carboxyl, C₁ to C₄ alkyl, C₁ to C₄ alkoxy, or halogen; and
Y is CH═CH₂ or —CH₂CH₂Y¹, wherein Y¹ is hydroxyl or a group which can be eliminated under the action of alkali.

32. A process for preparing the diaminopyridine of claim 31 comprising reacting a 2,6-dichloropyridine of formula (3): 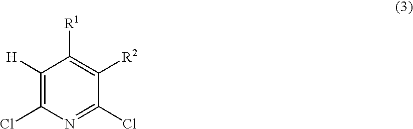
wherein
R¹ is C₁ to C₄ alkyl; and
R² is cyano, carbamoyl, or sulfomethyl;
with one mole equivalent of an amine of formula (4): 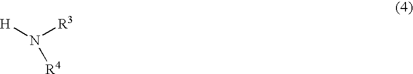
wherein
R³ and R⁴
are, independently of one another, hydrogen, C₁ to C₁₈ alkyl, or C₄ to C₇ cycloalkyl, wherein said alkyl and cycloalkyl is optionally substituted by hydroxyl and/or sulfo, and wherein, except for methyl, one or more nonadjacent carbon atoms is optionally replaced by oxygen, sulfone, sulfonyl (—SO₂—), or NR⁷, wherein R⁷ is hydrogen or C₁ to C₄ alkyl; or R³ and R⁴ are part of heterocyclic groups;
to give an isomer mixture of the monochloropyridine of formula (5): 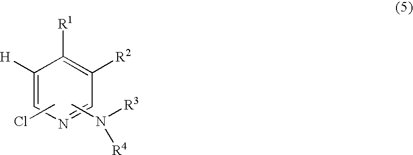
and reacting said monochloropyridine of formula (5) with an amine of formula (7): 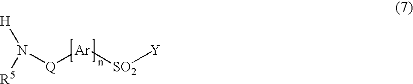
wherein
R⁵ are, independently of one another, hydrogen, C₁ to C₁₈ alkyl or C₄ to C₇ cycloalkyl, wherein said alkyl and cycloalkyl is optionally substituted by hydroxyl, sulfo, sulfato, or vinylsulfonyl and wherein, except for methyl, one or more nonadjacent carbon atoms is optionally replaced by oxygen or N>⁷, wherein R⁷ is hydrogen or C₁ to C₄ alkyl; or R³ and R⁴ are part of heterocyclic groups;
Q is C₁ to C₁₈ alkylene optionally substituted by C₁ to C₄ alkyl, halogen, or hydroxyl and wherein, except for methylene, one or more nonadjacent carbon atoms is optionally replaced by oxygen or NR⁷;
n is 0 or 1;
Ar is a phenylene or naphthylene radical optionally substituted by sulfo, carboxyl, C₁ to C₄ alkyl, C₁ to C₄ alkoxy, or halogen; and
Y is —CH₂CH₂OH;
or
reacting said monochloropyridine of formula (5) with an amine of formula (8): 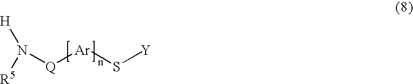
wherein
R⁵ is hydrogen, C₁ to C₁₈ alkyl or C₄ or C₇ cycloalkyl, wherein said alkyl and cycloalkyl are optionally substituted by hydroxyl and/or sulfo, and wherein, except for methyl, one or more nonadjacent carbon atoms is optionally replaced by oxygen or NR⁷, wherein R⁷ is hydrogen or C₁ to C₄ alkyl;
Q is C₁ to C₁₈ alkylene optionally substituted by C₁ to C₄ alkyl, halogen, or hydroxyl, and wherein, except for methylene, one or more nonadjacent carbon atoms is optionally replaced by oxygen or NR⁷;
n is 0 or 1;
Ar is a phenylene or naphthylene radical optionally substituted by sulfo, carboxyl, C₁ to C₄ alkyl, C₁ to C₄ alkoxy, or halogen; and
Y is —CH₂CH₂OH;
followed by oxidizing the intermediate obtained from the reaction of compound (5) with compound (8) and having formula (10): 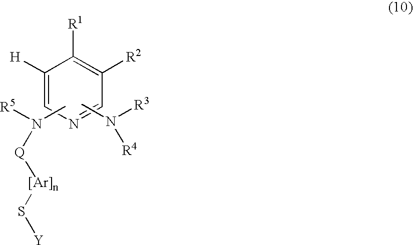
followed by reaction with the corresponding acid derivative or corresponding acid to give the desired diaminopyridine of formula (2), which can be converted conventionally to the corresponding vinyl compound.

33. A process for preparing the diaminopyridine of claim 31 comprising reacting a 2,6-dichloropyridine of formula (3): 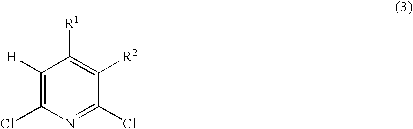
wherein
R¹ is C₁ to C₄ alkyl; and
R² is cyano, carbamoyl, or sulfomethyl;
with one mole equivalent of an amine of the formula (8): 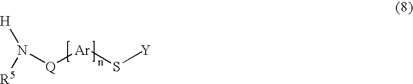
wherein
R⁵ is hydrogen, C₁ to C₁₈ alkyl, or C₄ or C₇ cycloalkyl, wherein said alkyl and cycloalkyl are optionally substituted by hydroxyl and/or sulfo, and wherein, except for methyl, one or more nonadjacent carbon atoms is optionally replaced by oxygen or NR⁷, wherein R⁷ is hydrogen or C₁ to C₄ alkyl;
Q is C₁ to C₁₈ alkylene optionally substituted by C₁ to C₄ alkyl, halogen, or hydroxyl, and wherein, except for methylene, one or more nonadjacent carbon atoms is optionally replaced by oxygen or NR⁷;
n is 0 or 1;
Ar is a phenylene or naphthylene radical optionally substituted by sulfo, carboxyl, C₁ to C₄ alkyl, C₁ to C₄ alkoxy, or halogen; and
Y is —CH₂CH₂OH;
to form the corresponding monochloropyridine derivative of formula (9); 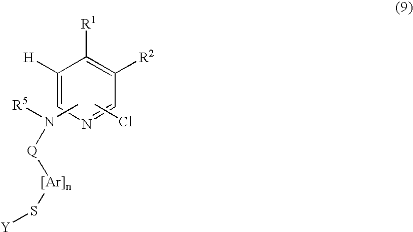
followed by oxidizing the compound of formula (9) to form the monochloropyridine of formula (6): 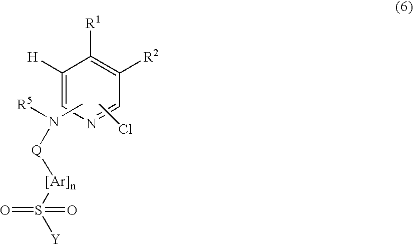
and further reacting the monochloropyridine of formula (6) with an amine of formula (4); 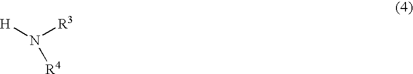
wherein
R³ and R⁴
are, independently of one another, hydrogen, C₁ to C₁₈ alkyl, or C₄ to C₇ cycloalkyl, wherein said alkyl or cycloalkyl is optionally substituted by hydroxyl and/or sulfo, and wherein, except for methyl, one or more nonadjacent carbon atoms is optionally replaced by oxygen, sulfone, sulfonyl (—SO₂—), or NR⁷, wherein R⁷ is hydrogen or C₁ to C₄ alkyl; or R³ and R⁴ are part of heterocyclic groups;
followed by reaction with the corresponding acid derivative or corresponding acid to give the desired diaminopyridine of formula (2), which can be converted conventionally to the corresponding vinyl compound.

34. A process for preparing the diaminopyridine of claim 31 comprising reacting a 2,6-dichloropyridine of formula (3): 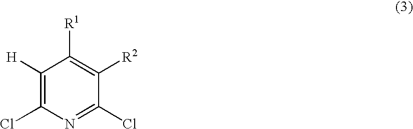
wherein
R¹ is C₁ to C₄ alkyl; and
R² is cyano, carbamoyl, or sulfomethyl;
with one mole equivalent of an amine of formula (8): 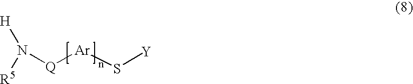
wherein
R⁵ is hydrogen, C₁ to C₁₈ alkyl, or C₄ or C₇ cycloalkyl, wherein said alkyl and cycloalkyl are optionally substituted by hydroxyl and/or sulfo, and wherein, except for methyl, one or more nonadjacent carbon atoms is optionally replaced by oxygen or NR⁷, wherein R⁷ is hydrogen or C₁ to C₄ alkyl;
Q is C₁ to C₁₈ alkylene optionally substituted by C₁ to C₄ alkyl, halogen, or hydroxyl, and wherein, except for methylene, one or more nonadjacent carbon atoms is optionally replaced by oxygen or NR⁷;
n is 0 or 1;
Ar is a phenylene or naphthylene radical optionally substituted by sulfo, carboxyl, C₁ to C₄ alkyl, C₁ to C₄ alkoxy, or halogen; and
Y is —CH₂CH₂OH;
to form the corresponding monochloropyridine derivative of formula (9): 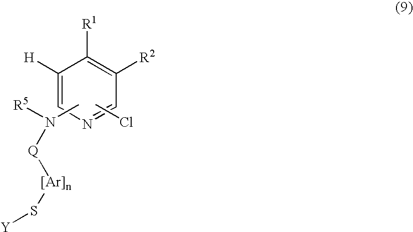
and further reacting the monochloropyridine of formula (9) with an amine of formula (4): 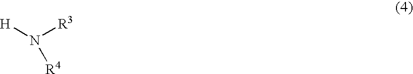
wherein
R³ and R⁴
are, independently of one another, hydrogen, C₁ to C₁₈ alkyl, or C₄ to C₇ cycloalkyl, wherein said alkyl or cycloalkyl is optionally substituted by hydroxyl and/or sulfo, and wherein, except for methyl, one or more nonadjacent carbon atoms is optionally replaced by oxygen, sulfonyl (—SO₂—), or NR⁷, wherein R⁷ is hydrogen or C₁ to C₄ alkyl; or R³ and R⁴ are part of heterocyclic groups;
followed by oxidizing the intermediate obtained from the reaction of compound (5) with compound (8) and having formula (10): 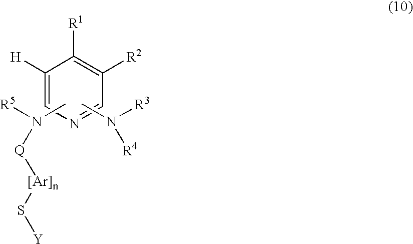
followed by reaction with the corresponding acid derivative or corresponding acid to give the desired diaminopyridine of formula (2), which can be converted conventionally to the corresponding vinyl compound.

35. A process for preparing the diaminopyridine of claim 31 comprising reacting a 2,6-dichloropyridine of formula (3): 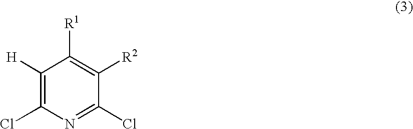
wherein
R¹ is C₁ to C₄ alkyl; and
R² is cyano, carbamoyl, or sulfomethyl;
with one mole equivalent of an amine of formula (7): 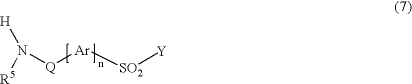
wherein
R⁵ are, independently of one another, hydrogen, C₁ to C₁₈ alkyl, or C₄ to C₇ cycloalkyl, wherein said alkyl and cycloalkyl is optionally substituted by hydroxyl, sulfo, sulfato, or vinylsulfonyl and wherein, except for methyl, one or more nonadjacent carbon atoms is optionally replaced by oxygen or NR⁷, wherein R⁷ is hydrogen or C₁ to C₄ alkyl; or R³ and R⁴ are part of heterocyclic groups;
Q is C₁ to C₁₈ alkylene optionally substituted by C₁ to C₄ alkyl, halogen, or hydroxyl and wherein, except for methylene, one or more nonadjacent carbon atoms is optionally replaced by oxygen or NR⁷;
n is 0 or 1;
Ar is a phenylene or naphthylene radical optionally substituted by sulfo, carboxyl, C₁ to C₄ alkyl, C₁ to C₄ alkoxy, or halogen; and
Y is —CH₂CH₂OH;
to form the monochloropyridine of formula (6): 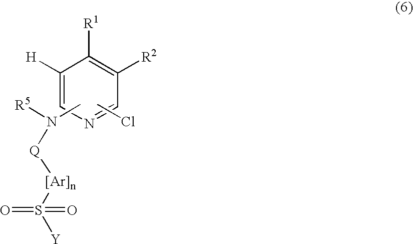
and further reacting the monochloropyridine of formula (6) with an amine of formula (4): 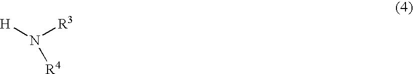
R³ and R⁴
are, independently of one another, hydrogen, C₁ to C₁₈ alkyl, or C₄ to C₇ cycloalkyl, wherein said alkyl or cycloalkyl is optionally substituted by hydroxyl and/or sulfo, and wherein, except for methyl, one or more nonadjacent carbon atoms is optionally replaced by oxygen, sulfone, sulfonyl (—SO₂—), or NR⁷, wherein R⁷ is hydrogen or C₁ to C₄ alkyl; or R³ and R⁴ are part of heterocyclic groups;
followed by reaction with the corresponding acid derivative or corresponding acid to give the desired diaminopyridine of formula (2).

36. A process for preparing the diaminopyridine of claim 31 comprising reacting a 2,6-dichloropyridine of formula (3): 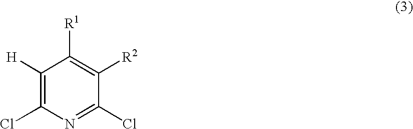
wherein
R¹ is C₁ to C₄ alkyl; and
R² is cyano, carbamoyl, or sulfomethyl;
with two mole equivalents of an amine of formula (8): 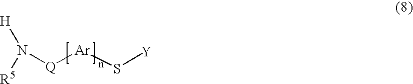
wherein
R⁵ is hydrogen, C₁ to C₁₈ alkyl or C₄ or C₇ cycloalkyl, wherein said alkyl and cycloalkyl are optionally substituted by hydroxyl and/or sulfo, and wherein, except for methyl, one or more nonadjacent carbon atoms is optionally replaced by oxygen or NR⁷, wherein R⁷ is hydrogen or C₁ to C₄ alkyl;
Q is optionally substituted C₁ to C₁₈ alkylene, and wherein, except for methylene, one or more nonadjacent carbon atoms is optionally replaced by oxygen or NR⁷;
n is 0;
Ar is a phenylene or naphthylene radical optionally substituted by sulfo, carboxyl, C₁ to C₄ alkyl, C₁ to C₄ alkoxy, or halogen; and
Y is —CH₂CH₂OH;
subsequently oxidizing the resultant intermediate;
followed by reaction with the corresponding acid derivative or corresponding acid to give the desired diaminopyridine of formula (2), which can be converted conventionally to the corresponding vinyl compound.

37. A process for preparing the diaminopyridine of claim 31 comprising reacting a 2,6-dichloropyridine of formula (3): 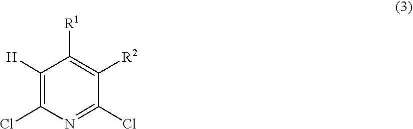
R¹ is C₁ to C₄ alkyl; and
R² is cyano, carbamoyl, or sulfomethyl;
with two mole equivalents of an amine of formula (7): 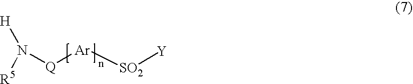
wherein
R⁵ is hydrogen, C₁ to C₁₈ alkyl, or C₄ or C₇ cycloalkyl, wherein said alkyl and cycloalkyl are optionally substituted by hydroxyl and/or sulfo, and wherein, except for methyl, one or more nonadjacent carbon atoms is optionally replaced by oxygen or NR⁷, wherein R⁷ is hydrogen or C₁ to C₄ alkyl;
Q is optionally substituted C₁ to C₁₈ alkylene, and wherein, except for methylene, one or more nonadjacent carbon atoms is optionally replaced by oxygen or NR⁷;
n is 0;
Ar is a phenylene or naphthylene radical optionally substituted by sulfo, carboxyl, C₁ to C₄ alkyl, C₁ to C₄ alkoxy, or halogen; and
Y is —CH₂CH₂OH;
followed by reaction with the corresponding acid derivative or corresponding acid to give the desired diaminopyridine of formula (2), which can be converted conventionally to the corresponding vinyl compound.

38. The process of claim 32, wherein said oxidation takes place in water, an organic solvent, or a mixture of water and organic solvent and in the presence of a catalyst.

39. The process of claim 32, wherein the monochloropyridine of formula (5) is prepared in water, an organic solvent, or without solvent and at temperatures from 30 to 150° C. in the presence of a base.

40. The process of claim 33, wherein the monochloropyridines of formulae (6) and (9) are prepared in water, an organic solvent, or without solvent and at temperatures from 30 to 150° C. in the presence of a base.

41. The process of claim 32, wherein the derivatization takes place in an organic solvent or without solvents in the corresponding acid or corresponding acid derivative.

42. A process for preparing the reactive dyes of claim 24 comprising reacting the corresponding diazonium salts with a coupling component of the formula (2): 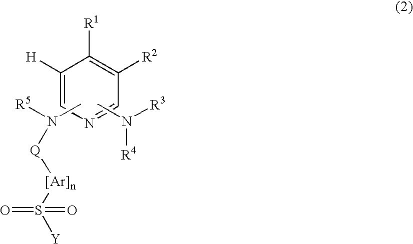
wherein
R¹ is C₁ to C₄alkyl;
R² is cyano, carbamoyl, or sulfomethyl;
R³, R⁴, and R⁵
are, independently of one another, hydrogen, C₁ to C₁₈ alkyl, or C₄ to C₇ cycloalkyl, wherein said alkyl or cycloalkyl is optionally substituted by hydroxyl, sulfo, sulfato, or vinylsulfonyl, and wherein, except for methyl, one or more nonadjacent carbon atoms is optionally replaced by oxygen or NR⁷, or R³ and R⁴ are part of heterocyclic groups;
R⁷ is hydrogen or C₁ to C₄ alkyl;
Q is C₁ to C₁₈ alkylene optionally substituted by C₁ to C₄ to alkyl, halogen, or hydroxyl and wherein, except for methylene, one or more nonadjacent carbon atoms is optionally replaced by oxygen or NR⁷,
n is 0 or 1;
Ar is a phenylene or naphthylene radical optionally substituted by sulfo, carboxyl, C₁ to C₄ alkyl, C₁ to C₄ alkoxy, or halogen; and
Y is —CH═CH₂ or CH₂CH₂Y¹, wherein Y¹ is hydroxyl or a group which can be eliminated under the action of alkali; and
wherein said corresponding diazonium salts are prepared by conventional diazotization of amines of formula D-NH₂.

43. A dye formulation comprising one or more reactive dyes of claim 23 for dyeing and printing carboxamido- and/or amino- and/or hydroxyl-containing material.

44. An aqueous printing ink for textile printing by the ink-jet method comprising one or more reactive dyes of claim 23 in amounts in the range of from 0.01% to 40% by weight, based on the total ink weight.
